(19) United States
(12) Patent Application Publication
(10) Pub. No.: US 2023/0201164 A1
(43) Pub. Date: Jun. 29, 2023

(54) PHARMACEUTICAL COMPOSITION COMPRISING BENZIMIDAZOLE DERIVATIVE COMPOUND

(71) Applicant: HK INNO.N CORPORATION, Cheongju-si Chungcheongbuk-do (KR)

(72) Inventors: Suchul LEE, Gwacheon-si Gyeonggi-do (KR); Eun Kyung JEON, Hwaseong-si Gyeonggi-do (KR); Young Dae CHO, Osan-Si Gyeonggi-do (KR); Sung Ah LEE, Guri-si Gyeonggi-do (KR); Dong Hyun KIM, Suwon-si Gyeonggi-do (KR); Myeongjoong KIM, Anyang-si Gyeonggi-do (KR); So Hyun JOO, Suwon-si Gyeonggi-do (KR); Bong Tae KIM, Hanam-si Gyeonggi-do (KR)

(21) Appl. No.: 17/802,054

(22) PCT Filed: Feb. 26, 2021
(86) PCT No.: PCT/IB2021/051609

§ 371 Date: Aug. 24, 2022

(30) Foreign Application Priority Data
Feb. 27, 2020 (KR) 10-2020-0024657
Apr. 8, 2020 (KR) 10-2020-0043047

(51) Int. Cl. A61K 31/4178; A61K 9/50

(52) U.S. Cl. CPC A61K 31/4178; A61K 9/5026

(57) ABSTRACT

The present disclosure relates to a pharmaceutical composition containing a benzimidazole derivative compound. Specifically, the present disclosure relates to a formulation capable of maintaining a sustained blood concentration of the benzimidazole derivative compound.

### TECHNICAL FIELD

The present disclosure relates to a pharmaceutical composition containing a benzimidazole derivative compound. Specifically, the present disclosure relates to a formulation capable of maintaining a sustained blood concentration of the benzimidazole derivative compound.

### BACKGROUND

Tegoprazan is the world's first potassium-competitive acid blocker (P-CAB), has a mechanism similar to that of an acid pump antagonist (APA), and blocks gastric acid secretion by competing with potassium ions for binding to the enzyme H⁺/K⁺-ATPase (proton pump) that secretes H⁺ ions, which are a component of gastric acid, from the gastric parietal cells into the gastric lumen. Since tegoprazan is not a prodrug such as a proton pump inhibitor (PPI), it does not require an activation process, and thus acts not only on an active proton pump but also on an inactive proton pump. Thus, tegoprazan has the advantages of exhibiting its effect rapidly and reaching the maximum effect within one hour.

Meanwhile, in general, in order for a drug to exhibit an expected effect, the blood concentration of the drug needs to be maintained at a certain level or higher. To maintain the blood concentration of the drug, a patient is required to take the prescribed drug repeatedly according to a certain schedule. In this case, taking the drug frequently decreases the patient's medication compliance, and as a result, there are many cases where the expected therapeutic effect is not obtained. Thus, in a disease for which a drug needs to be taken for a long period of time or the blood concentration of the drug at a time when the patient cannot take the drug needs to be maintained at a certain level or higher, the frequency and method of taking the drug is also an important factor to be considered for increasing the therapeutic effect of the drug. Accordingly, there is a need to develop a formulation capable of maintaining a therapeutically effective blood concentration of a drug because there is no problem in the absorption rate of the drug while modifying the release of the drug.

### DISCLOSURE

### Technical Problem

An object of the present disclosure is to provide a modified-release pharmaceutical composition containing: tegoprazan, an optical isomer thereof, a pharmaceutically acceptable salt thereof, a hydrate or solvate thereof, or a mixture thereof as an active ingredient; and a release modifying agent.

An object of the present disclosure is to provide a modified-release pharmaceutical composition including: a core containing, tegoprazan, an optical isomer thereof, a pharmaceutically acceptable salt thereof, a hydrate or solvate thereof, or a mixture thereof as an active ingredient; and a release modifying agent-containing layer formed on the core.

An object of the present disclosure is to provide a capsule filled with the modified-release pharmaceutical composition.

An object of the present disclosure is to provide a tablet including the modified-release pharmaceutical composition.

An object of the present disclosure is to provide a formulation including: a modified-release first pharmaceutical composition which contains, tegoprazan, an optical isomer thereof, a pharmaceutically acceptable salt thereof, a hydrate or solvate thereof, or a mixture thereof as an active ingredient; and a second pharmaceutical composition which contains tegoprazan, an optical isomer thereof, a pharmaceutically acceptable salt thereof, a hydrate or solvate thereof, or a mixture thereof as an active ingredient, and releases the active ingredient immediately.

### Technical Solution

Terms that are not specifically defined in the present specification will be understood as having the same meaning as commonly used in the art to which the present disclosure pertains. In addition, singular expressions include plural expressions, and plural expressions include singular expressions, unless specified otherwise in the context thereof.

In the present specification, terms such as first and second are only used for classification, and are not intended to specify an order or location.

In the present specification, items are only arbitrarily divided for convenience of description of the specification, and the content of any one item should not be interpreted as being a subordinate to the item.

In the present specification, a multilayered-tablet may be a tablet in which one or more layers surrounding a core are positioned on the core, and the one or more layers may be coating layers and/or matrix layers. For example, the multilayered tablet may be a tab-in-tab as illustrated in FIG. 1A.

Also, in the present specification, multilayered tablet may be in a form in which one or more layers are continuously stacked as illustrated in FIG. 1B. For example, the multilayered-tablet may be a bilayer tablet, a trilayer tablet, etc.

In the present specification, tegoprazan is a compound represented by the following Formula I, and has a chemical name of (S)-4-((5,7-difluorochroman-4-yl)oxy)-N,N,2-trimethyl-1H-benzo[d]imidazole-6-carboxamide.

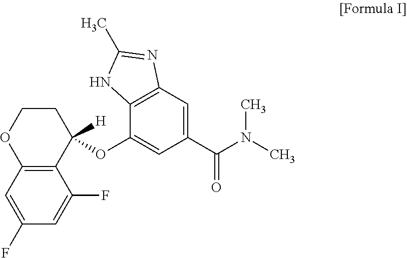 [SMILES: Cc1nc2cc(C(=O)N(C)C)cc(O[C@H]3CCOc4cc(F)cc(F)c43)c2[nH]1]

In the present specification, the term “tegoprazan” may refer to tegoprazan, an optical isomer thereof, a pharmaceutically acceptable salt thereof, a hydrate or solvate thereof, or a mixture thereof. In addition, in the present specification, the term “tegoprazan” may be used interchangeably with the term “active ingredient”.

In the present invention, the “pharmaceutically acceptable salt” may be an acid addition salt or a base addition salt. The acid addition salt may be prepared from an acid that forms a non-toxic salt, and examples thereof include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Examples of the base addition salt include alkali metal salts such as lithium salts, sodium salts and potassium salts; alkaline earth metal salts such as calcium salts and magnesium salts; ammonium salts; and organic base salts such as triethylamine salts, diisopropylamine salts, or cyclohexylamine salts. The base addition salt may be specifically an alkali metal salt, more specifically a sodium salt.

For a review on suitable salts, see “Handbook of Pharmaceutical Salts: Properties, Selection, and Use” by Stahl and Wermuth (Wiley-VCH, 2002). If appropriate, a pharmaceutically acceptable salt of the compound of Formula I may be easily produced by mixing a solution of the compound of formula I with the desired acid or base. The salt may be precipitated from the solution and collected by filtration, or the salt may be recovered by evaporating the solvent. The degree of ionization of the salt may vary from a fully ionized state to an almost non-ionized state.

The term “immediate release (IR)” as used herein means that the active ingredient is released immediately or within a short time after administration.

As used herein, the term “controlled release (CR) or modified-release (MR)” means that the release of the drug is controlled so that the active ingredient is released at a specific location in the gastrointestinal tract or after a certain time after taking the drug, or is released in the gastrointestinal tract in a sustained manner over a long period of time, or is released in a sustained manner over a long period of time while being released at a specific location in the gastrointestinal tract or after a certain time after taking the drug. That is, in the present specification, the term “modified release” or “controlled release” may include delayed release and/or extended release or sustained release. Specifically, “modified release” or “controlled release” may be either delayed release in which the drug is released after a certain time after taking the drug; or sustained release in which the drug is slowly released over a long period of time over a certain time after taking the drug; or delayed release and sustained release in which the drug is slowly released over a certain period of time while being released after a certain time after taking the drug. For example, the delayed release may mean that the drug starts to be released in an environment other than gastric juice after taking the drug, and the sustained release may mean that the drug is continuously released in a region ranging from the gastric juice environment to the intestinal environment after taking the drug, or that the drug is released in a sustained manner after the drug starts to be released in an environment other than gastric juice. As used herein, the terms “modified-release” and “controlled release” may be interchangeable with each other.

Modified-Release Pharmaceutical Composition

The present disclosure provides a modified-release pharmaceutical composition containing: tegoprazan, an optical isomer thereof, a pharmaceutically acceptable salt thereof, a hydrate or solvate thereof, or a mixture thereof as an active ingredient; and a release modifying agent.

In examples of the present disclosure, the release modifying agent may include at least one selected from the group consisting of a sustained-release agent and an enteric agent.

In one embodiment, the modified-release pharmaceutical composition of the present disclosure may contain a sustained-release agent.

In another embodiment, the modified-release pharmaceutical composition of the present disclosure may contain an enteric agent.

In another embodiment, the modified-release pharmaceutical composition of the present disclosure may contain a sustained-release agent and an enteric agent.

The pharmaceutical composition of the present disclosure is a modified-release pharmaceutical composition with modified-release of the tegoprazan. For example, tegoprazan may be delayed-released, or sustained-released, or delayed-released and sustained-released from the pharmaceutical composition. Specifically, the pharmaceutical composition of the present disclosure may reach the intestines (e.g., duodenum, small intestine, etc.) after passing through the gastric juice environment and release tegoprazan (that is, delayed release), or may be continuously released starting with the gastric juice environment over a long period of time, or may reach the intestines after passing through the gastric juice environment and start to release tegoprazan (delayed release) and release tegoprazan in a sustained manner over a long period of time. Thus, the modified-release pharmaceutical composition of the present disclosure may maintain a high blood concentration of the active ingredient tegoprazan until a certain time after taking the drug, and thus may significantly improve the patient's medication compliance. Specifically, the composition of the present disclosure allows the tegoprazan to be released after passing through the gastric juice environment, or to be sustainedly released in a region ranging from the gastric juice to the intestinal environment, or to start to be released after passing through the gastric juice environment and to be sustainedly released. Thus, the composition of the present disclosure may exhibit a drug effect even after a certain time after taking the drug, and thus may significantly improve the patient's medication compliance. In addition, the composition of the present disclosure may exhibit an excellent drug effect for a long time even at a low dose, and thus minimize side effects and maximize the drug effect.

The modified-release pharmaceutical composition may exhibit excellent dissolution even in an environment having a higher pH than the gastric juice environment, and as a result, exhibit excellent dissolution in environments such as intestinal fluid environments, for example, duodenum and small intestine. In addition, it may modify drug release so that a therapeutic blood concentration of tegoprazan may be achieved without lowering the dissolution rate thereof even in case that tegoprazan is either released in a delayed manner or released in a sustained manner after being released in a delayed released.

In examples of the present disclosure, the modified-release pharmaceutical composition of the present disclosure may contain a particle containing, tegoprazan, an optical isomer thereof, a pharmaceutically acceptable salt thereof, a hydrate or solvate thereof, or a mixture thereof as an active ingredient.

In the present disclosure, the term “particle” may be used interchangeably with the term “tegoprazan-containing particle”.

In examples of the present disclosure, the particle may be a pellet, a tablet, or a granule.

In case that the particle is a pellet, the pellet may include: an inert particle; and an active ingredient layer, which contains tegoprazan, an optical isomer thereof, a pharmaceutically acceptable salt thereof, a hydrate or solvate thereof, or a mixture thereof as an active ingredient, formed on the inert particle.

The modified-release pharmaceutical composition may include a release modifying agent inside and/or outside the active ingredient layer. For example, the modified-release pharmaceutical composition may include a release modifying agent inside the active ingredient layer, or may include a release modifying agent layer formed on the active ingredient layer, or may include both a release modifying agent in the active ingredient layer and a modified-release release layer formed on the active ingredient layer.

The release modifying agent may be a sustained-release agent and/or an enteric agent. For example, where the composition includes the release modifying agent both inside and outside the active ingredient layer, the release modifying agent included in the active ingredient layer and the release modifying agent included outside the active ingredient layer may be the same as or different from each other. Specifically, a sustained-release agent, an enteric agent, or both may be included in the active ingredient layer, and a sustained-release agent, an enteric agent, or both may be included outside the active ingredient layer, wherein the agents included inside and outside the active ingredient layer may be independent of each other.

In examples of the present disclosure, a sustained-release agent may be included inside the active ingredient layer. In other examples of the present disclosure, a sustained-release agent may be included outside the active ingredient layer. In this case, the sustained-release agent may be included in the sustained-release agent-containing layer formed on the active ingredient layer. In still other examples of the present disclosure, a sustained-release agent may be included inside and outside the active ingredient layer. In other examples of the present disclosure, a sustained-release agent may be included inside the active ingredient layer, and an enteric agent may be included outside the active ingredient layer. In still other examples of the present disclosure, a sustained-release agent may be included inside the active ingredient layer, and a sustained-release agent and an enteric agent may be included outside the active ingredient layer, and in this case, a sustained-release agent layer formed on the active ingredient layer and an enteric agent layer formed on the sustained-release agent layer may be included outside the active ingredient layer.

In an embodiment, the pellet may include an inert particle and an active ingredient layer formed on the inert particle, wherein the active ingredient layer may include a first release modifying agent. In another embodiment, the pellet may include a second release modifying agent layer including a second release modifying agent formed on the active ingredient layer including the first release modifying agent. Or, in still another embodiment, the pellet may include: an inert particle; an active ingredient layer formed on the inert particle; and the first release modifying agent layer formed on the active ingredient layer.

In the above embodiments, the first release modifying agent may be a sustained-release agent, and the second release modifying agent may be a sustained-release agent or an enteric agent. In one example, the second release modifying agent may be an enteric agent. In other examples, the second release modifying agent may be a sustained-release agent, and in this case, the pellet may include a third release modifying agent layer formed on the second release modifying agent layer, and the third release modifying agent layer may include an enteric agent.

The active ingredient layer and the release modifying agent layer may each independently include a pharmaceutically acceptable additive. The pharmaceutically acceptable additive may include, but is not limited to, for example, an anti-adhesion agent, a plasticizer, a surfactant, a disintegrant, and an excipient. The content and type of the pharmaceutically acceptable additive may be appropriately selected by a person skilled in the art.

In case that the pellet includes a plurality of release modifying agent layers on the active ingredient layer, an additional layer may be included between the layer and the layer. The additional layer may facilitate coating of a subsequent layer, or function to prevent the components contained in the two layers from coming into direct contact with each other to interact with each other or result in reduction in stability. The term “additional layer” may be used interchangeably with the term “isolation layer” in the present disclosure.

The term “inert particle” as used herein may refer to a pharmaceutical additive that is a material in a regular or irregular form that does not include a material having pharmacological activity. In the present disclosure, the inert particle may be used alone, or may be mixed with an active ingredient and/or other pharmaceutically acceptable additives, and may function as a seed for the coating of a layer that is formed in the pharmaceutical composition of the present disclosure.

In the examples of the present disclosure, the inert particle include, for example, any one or more selected from pharmaceutically acceptable inert substances such as white sugar, lactose, starch, mannitol, sucrose, dextrin, or microcrystalline cellulose, and preferably include sucrose, but is not limited thereto.

In the examples of the present disclosure, the pharmaceutical composition may further include an organic acid. According to an example, the inert particle may include an organic acid or may be a material prepared with only an organic acid. According to another example, a layer containing an organic acid may be separately positioned in the core or outside the core.

The organic acid may serve to improve the solubility of the active ingredient. In case that the pharmaceutical composition contains an organic acid, the organic acid may serve to improve the dissolution of tegoprazan and increase the in vivo absorption rate thereof. For example, as the enteric agent-containing layer in the pharmaceutical composition of the present disclosure is completely or partially dissolved under weak alkaline conditions, tegoprazan is dissolved or suspended, and as the organic acid contained in the pharmaceutical composition is dissolved, the solubility of the suspended tegoprazan is increased, so that the dissolution and in vivo absorption rate thereof may be improved.

The organic acid may be, for example, any one or more selected from among tartaric acid, fumaric acid, succinic acid, citric acid, malic acid, glutamic acid and aspartic acid. Specifically, the organic acid may be any one or more selected from among tartaric acid, fumaric acid, succinic acid, and citric acid. More specifically, the organic acid may be tartaric acid, but is not limited thereto. In addition, in the present disclosure, the organic acid may include a hydrate or salt form.

In the examples of the present disclosure, the weight ratio between the inert particle and the active ingredient included in the core may be 5:1 to 1:5, specifically, 3:1 to 1:3, more specifically 1.5:1 to 1:1.5, even more specifically 1:1, but is not limited thereto.

In examples of the present disclosure, the active ingredient layer may further include a pharmaceutically acceptable additive. For example, the active ingredient layer may include povidone, polyethylene glycol, talc, polysorbate, or a mixture thereof.

In the present disclosure, the inert particle may be prepared by a conventional preparation method such as direct compression, compression of anhydrous, wet or sintered granules, extrusion and subsequent rounding off, wet or dry granulation, or direct pelletizing. In particular, in case that the inert particle is a pellet, it may be prepared by a pan method on a pelletizing plate or extrusion/rounding off, but is not limited thereto.

In case that the particle is a granule, the granule may be a granule prepared from a mixture of the active ingredient and a pharmaceutically acceptable additive. In this case, the granule may be a wet granule or a dry granule.

In examples of the present disclosure, the granule may include a release modifying agent inside and outside the granule, and the release modifying agent may be a sustained-release agent and/or an enteric agent. In case that a release modifying agent is included inside the granule, the granule may be formed from a mixture of the active ingredient and a release modifying agent. In case that a release modifying agent is included outside the granule, the release modifying agent may be included outside the granule (on the granule) containing the active ingredient; or the granule containing the active ingredient and the release modifying agent. In case that the granule includes release modifying agents both inside and outside the granule, the release modifying agent included inside the granule and the release modifying agent included outside the granule may be the same as or different from each other. Specifically, a sustained-release agent, an enteric agent, or both may be included inside the granule, and a sustained-release agent, an enteric agent, or both may be included outside the granule, and in this case, the release modifying agents included inside and outside the granule may be independent of each other.

In examples of the present disclosure, a sustained-release agent may be included inside the granule. In other examples of the present disclosure, a sustained-release agent may be included outside the granule. In still other examples of the present disclosure, a sustained-release agent may be included inside and outside the granule. In other examples of the present disclosure, a sustained-release agent may be included inside the granule, and an enteric agent may be included outside the granule. In still other examples of the present disclosure, a sustained-release agent and an enteric agent may be included the outside the granule including or not including a sustained-release agent therein, and in this case, the granule may include a sustained-release agent layer formed on the outside of the granule and an enteric agent layer formed on the sustained-release agent layer.

In case that the particle is a tablet, the tablet may be in the form of a tablet produced by tableting a granule, a pellet or a mixture thereof, which contains the active ingredient, and a pharmaceutically acceptable additive. In this case, the granule may be a wet granule or a dry granule, and the pellet may include an active ingredient-containing coating layer in an inert particle.

The tablet may include a release modifying agent inside the tablet and/or outside the tablet (on the tablet), and description of the release modifying agent may be the same as described above with respect to the release modifying agent inside and/or outside each of the pellet and the granule, unless there are contradictions.

In examples of the present disclosure, the tablet may include a sustained-release agent inside the tablet. In other examples of the present disclosure, a sustained-release agent may be included outside the tablet (on the tablet). In still other examples of the present disclosure, a sustained-release agent may be included inside and outside the tablet. In other examples of the present disclosure, a sustained-release agent may be included inside the tablet, and an enteric agent may be included outside the tablet. In still other examples of the present disclosure, a sustained-release agent and an enteric agent may be included outside of a tablet including or not including a sustained-release agent, and in this case, the tablet may include a sustained-release agent layer formed on the tablet and an enteric agent layer formed on the sustained-release agent layer.

In the present disclosure, the active ingredient tegoprazan may exist in a crystalline or amorphous form.

In examples of the present disclosure, the present disclosure provides a modified-release pharmaceutical composition including: a core containing, tegoprazan, an optical isomer thereof, a pharmaceutically acceptable salt thereof, a hydrate or solvate thereof, or a mixture thereof as an active ingredient; and a release modifying agent-containing layer formed on the core.

The release modifying agent-containing layer may include a sustained-release agent-containing layer and/or an enteric agent-containing layer. In the present disclosure, the terms “release modifying agent-containing layer”, “sustained-release agent-containing layer”, and “enteric agent-containing layer” may be used interchangeably with the term “release modifying agent layer”, “sustained-release agent layer”, and “enteric agent layer”, respectively.

In the present specification, the term “core” refers to a part constituting the center or core of the pharmaceutical composition. The core may be completely coated by the coating layer to be formed later and located in the center of the pharmaceutical composition, but a portion of the core may not be coated within a range in which the function thereof does not significantly differ from that of the completely coated core. The core may also be positioned to be biased to one side of the pharmaceutical composition.

The core may include a particle containing, tegoprazan, an optical isomer thereof, a pharmaceutically acceptable salt thereof, a hydrate or solvate thereof, or a mixture thereof as an active ingredient, or the core may be the particle.

The particle is substantially the same as described above with respect to the particle containing tegoprazan, unless there are contradictions.

Specifically, in examples of the present disclosure, the core may include: an inert particle; and an active ingredient-containing active ingredient layer formed on the inert particle.

The inert particle and the active ingredient layer are substantially the same as described above, unless there are contradictions.

According to examples of the present disclosure, the core may be a mixture of the active ingredient and a pharmaceutically acceptable additive. In this case, the active ingredient and the pharmaceutically acceptable additive may be present throughout the core, and for example, may be mixed in a single matrix form. According to an example, the core may be a granule prepared from a mixture of the active ingredient and a pharmaceutically acceptable additive. In this case, the granule may be a wet granule or a dry granule. According to another example, the core may be in the form of a core tablet prepared by tableting a granule, a pellet, or a mixture thereof, which contains the active ingredient, and a pharmaceutically acceptable additive. In this case, the granule may be a wet granule or a dry granule, and the pellet may include an active ingredient-containing coating layer on an inert particle.

The pharmaceutically acceptable additive may include, but is not limited to, for example, an anti-adhesion agent, a plasticizer, a surfactant, a disintegrant, and an excipient.

The core may contain a release modifying agent, specifically, the core may contain a sustained-release agent and/or an enteric agent. More specifically, the core may contain a sustained-release agent.

The modified-release pharmaceutical composition of the present disclosure may include a sustained-release agent.

In the present disclosure, the sustained-release agent may be a material capable of releasing the drug in a sustained manner for a predetermined period of time by lowering the release rate of the drug from the pharmaceutical composition of the present disclosure. In the present disclosure, the sustained-release agent may comprise a water insoluble and/or poorly water soluble material having a property sufficient to enable the sustained release of active ingredient, for example a viscosity sufficient to enable the sustained release of active ingredient, but not limited thereto. For example, in case the sustained-release agent in the present disclosure comprises water insoluble and/or poorly water soluble material, the water insoluble and/or poorly water soluble material may be used in combination with water soluble substance, but not limited thereto. In the present disclosure, the sustained-release agent may include one or more selected from among known sustained-release agents, for example, including, methacrylic acid copolymer, polyethylene oxide, cellulose acetate, copovidone, hydroxypropyl ethylcellulose, glyceryl distearate, methylcellulose, polyvinyl alcohol, ethyl cellulose, polyethylene glycol-polyvinyl alcohol copolymers, hydroxypropyl cellulose, hypromellose (hydroxypropyl methyl cellulose), microcrystalline cellulose, mannitol, sucrose, lactose, polyethylene glycol, polyvinyl pyrrolidone, sodium carboxymethylcellulose, pregelatinized starch, natural gum, synthetic gum, polyvinylpyrrolidone copolymers, povidone, gelatin, starch, highly dispersible silica, talc, or mixtures thereof, but not limited to. Specifically, in the present disclosure, the sustained-release agent may be, but is not limited to, polyvinyl alcohol, hydroxypropyl cellulose, a polyethylene glycol-polyvinyl alcohol copolymer, polyethylene oxide, methacrylic acid copolymer, hydroxypropyl methyl cellulose, ethyl cellulose, povidone, talc, or a mixture thereof. In examples of the present disclosure, the sustained-release agent may include at least one selected from the group consisting of polyvinyl alcohol, polyethylene oxide, methacrylic acid copolymer, hydroxypropyl methyl cellulose, ethyl cellulose, povidone, and talc. In other examples of the present disclosure, the sustained-release agent may include one or more selected from the group consisting of polyethylene oxide, methacrylic acid copolymer, polyvinyl alcohol, ethyl cellulose, povidone, and talc.

In examples of the present disclosure, the sustained-release agent may be included inside and/or outside the core. In case that the sustained-release agent is included inside the core, the core may be a mixture of the active ingredient, the sustained-release agent and a pharmaceutically acceptable additive, and the active ingredient, the sustained-release agent and the pharmaceutically acceptable additive may be present throughout the core, and for example, may be mixed in a single matrix form. For example, in case that the core is a pellet, the core may include a sustained-release agent in an active ingredient layer formed on an inert particle, and in case that the core is a granule, the core may include a sustained-release agent together with the active ingredient inside the granule, and in case that the core is a tablet, a sustained-release agent may be included inside the tablet. In case that the sustained-release agent is included outside the core, the sustained-release agent may be formed on the core so as to surround the core. For example, in case that the core is a pellet, the pellet may include a sustained-release agent (containing) layer formed on the active ingredient layer. In case that the core is a granule, the granule may include a sustained-release agent (containing) layer formed on the granule and surrounding the granule. In case that the core is a tablet, the tablet may include a sustained-release agent (containing) layer formed on the tablet.

The sustained-release agent layer may include a pharmaceutically acceptable additive, and may include, for example, talc, but is not limited thereto. The content and type of the pharmaceutically acceptable additive that is included in the sustained-release agent layer may be appropriately selected by a person skilled in the art.

In case that the modified-release pharmaceutical composition of the present disclosure includes a sustained-release agent layer, the sustained-release agent layer may be included in an amount of about 10 to 70 wt %, specifically, about 10 to 50 wt %, more specifically 10 to 40 wt %, even more specifically about 10 to 30 wt %, by weight based on the total weight of the composition.

The modified-release pharmaceutical composition of the present disclosure may contain an enteric agent.

In the present disclosure, the enteric agent refers to a material that does not dissolve in the stomach, but reaches and dissolves in the intestines, for example, duodenum, etc. Specifically, the enteric agent may be a material that does not dissolve in the gastric pH environment (pH 2 or less) and begins to dissolve in the intestinal pH environment (pH 5 to 7.5).

In the present disclosure, the enteric agent may be one or more selected from known enteric agents. For example, the enteric agent may be, but is not limited to, any one or more selected from the group consisting of ethyl cellulose, cellulose acetate, polyvinyl acetate, cellulose butyrate phthalate, cellulose hydrogen phthalate, cellulose propionate phthalate, polyvinyl acetate phthalate, cellulose acetate phthalate, cellulose acetate trimellitate, hydroxypropyl methyl cellulose phthalate, polyvinyl acetate, hydroxypropyl methyl acetate, dioxypropyl methyl cellulose succinate, carboxymethyl ethyl cellulose, hydroxypropyl methyl cellulose acetate succinate, and polymers thereof; Shellac; and acrylic acid, methacrylic acid, or esters thereof, or copolymer formed from thereof. Specifically, the copolymer formed from acrylic acid, methacrylic acid or esters thereof may be methacrylic acid-ethyl acrylate copolymer (e.g., Eudragit L30D-55 and L100-55), methacrylic acid copolymer L (e.g., Eudragit L100), methacrylic acid copolymer S (e.g., Eudragit S100), and methacrylic acid-methylacrylate-methylmethacrylate copolymer (e.g., Eudragit FS30D).

The term “methacrylic acid copolymer L” as used herein refers to an anionic copolymer containing methacrylic acid and methylmethacrylate in a ratio of about 1:1, and the IUPAC name thereof is poly(methacrylic acid-co-methyl methacrylate) 1:1.

The term “methacrylic acid copolymer S” as used herein refers to an anionic copolymer containing methacrylic acid and methylmethacrylate in a ratio of about 1:2, and the IUPAC name thereof is poly(methacrylic acid-co-methyl methacrylate) 1:2.

In the present disclosure, the enteric agent may be a pH-dependent enteric agent, and examples thereof include a methacrylic acid-ethyl acrylate copolymer soluble at pH 5.5 or higher, methacrylic acid copolymer L soluble at pH 6.0 or higher, and methacrylic acid copolymer S soluble at pH 7.0 or higher.

The term “pH dependent” as used herein means that the elution or dissolution of the enteric agent begins in an environment with a certain pH or higher.

The term “pH-dependent soluble” as used herein means that the enteric agent dissolves in an environment with a certain pH or higher.

According to one embodiment of the present disclosure, in case that a mixture of the enteric agents includes methacrylic acid copolymer L and S, methacrylic acid copolymer L and methacrylic acid copolymer S may be mixed at a weight ratio of, but not limited to, 1:3 to 0.2, specifically 1:1.5 to 1:0.4. According to another embodiment, in case that the mixture of the enteric agents includes a methacrylic acid-ethyl acrylate copolymer and methacrylic acid copolymer S, the methacrylic acid-ethyl acrylate copolymer and the methacrylic acid copolymer S may be mixed at a weight ratio of, but not limited to, 0.3:1 to 3:1, specifically, 0.5:1 to 2:1.

In case that the modified-release pharmaceutical composition of the present disclosure contains enteric agents at the above-described ratio, the active ingredient tegoprazan may be released in a delayed manner, so that tegoprazan may exhibit a sufficient drug effect even after a certain time after taking the same.

In the examples of the present disclosure, the enteric agent-containing layer may be soluble at pH 5.0 or higher, pH 5.5 or higher, pH 6.0 or higher, or pH 6.5 or higher. According to one embodiment of the present disclosure, the modified-release layer may be pH-dependent soluble at pH 5.5 or higher. According to another embodiment, the modified-release layer may be pH-dependent soluble at pH 6.0 or higher. According to still another embodiment, the modified-release layer may be pH-dependently soluble at pH 6.5 or higher. According to yet embodiment, the modified-release layer may be pH-dependent soluble at pH 7.0 or higher.

The term “insoluble” or “poorly soluble” as used herein refers to the property of any substance that does not dissolve or hardly dissolve in a solvent, and, on the contrary, the term “soluble” means that any substance dissolves well in a solvent. In the pharmaceutical composition of the present disclosure, the enteric agent-containing layer may be included in an amount of about 10 to 70 wt %, specifically about 10 to 50 wt %, more specifically 10 to 40 wt %, even more specifically about 10 to 30 wt %, based on the total weight of the composition.

In examples of the present disclosure, in case that the composition contains the enteric agent or the enteric agent-containing layer, it may have acid resistance. Specifically, the dissolution rate of the active ingredient in a dissolution medium with pH 1.2 may be less than 10% at 120 minutes, more specifically less than 5% at 120 minutes. On the other hand, the dissolution rate of the active ingredient in a dissolution medium with pH 5 or more may be 50% or higher, more preferably 60% or higher, within 360 minutes.

The term “acid resistance” used as used herein refers to a case where the dissolution of the active ingredient under an acidic condition is 10% or less as determined according to the guidelines for dissolution standards for oral drugs. In general, whether acid resistance is ensured may be determined by measuring whether the active ingredient is released for 2 hours under a low pH condition (generally pH 1 to 2).

In the present disclosure, the dissolution rate may be measured according to the Pharmacopoeia dissolution test method 1 (basket method) or dissolution test method 2 (paddle method). Specifically, the dissolution test method may be performed at a dissolution medium temperature of 36.5 to 37.5° C., a dissolution medium volume of 500 mL to 1000 mL, and a rotating speed of 50 rpm to 100 rpm. In the case of the dissolution test method 1, the rotating speed of the basket may preferably be 100 rpm, and in the case of the dissolution test method 2, the rotating speed of the paddle may be preferably 75 rpm.

The enteric agent-containing layer of the present disclosure may further contain a pharmaceutically acceptable additive. Examples of the additive that may further be contained include, but are not limited to, a binding agent, an anti-adhesion agent, a plasticizer, a surfactant, a disintegrant, and an excipient. One or more of the pharmaceutically acceptable additives may be contained in the modified-release layer, and the content and type thereof may be appropriately selected by a person skilled in the art. For example, the enteric agent-containing layer may contain, triethyl citrate, polysorbate, or a mixture thereof as a pharmaceutically acceptable additive. In the examples of the present disclosure, the pharmaceutical composition of the present disclosure may further include one or more additional layers containing only a pharmaceutically acceptable additive without the active ingredient. The additional layer may facilitate coating of a subsequent layer when the pharmaceutical composition is prepared by a method of forming a plurality of coating layers, or may function to prevent the components contained in two layers from coming into direct contact with each other to interact with each other or result in reduction in stability. The term “additional layer” may be used interchangeably with the term “separation layer” or “isolation layer” in the present disclosure.

According to examples of the present disclosure, the additional layer is positioned between the core and the release modifying agent-containing layer; and/or on the release modifying agent-containing layer. According to other examples of the present disclosure, in case that the pharmaceutical composition includes two or more release modifying agent-containing layers, the additional layer may be positioned between the first release modifying agent-containing layer and the second release modifying agent-containing layer; and/or on the second release modifying agent-containing layer.

For example, in case that the core of the pharmaceutical composition is a tablet, the pharmaceutical composition may further include an additional layer between the tablet and the release modifying agent-containing layer and/or on the release modifying agent-containing layer. In case that the core of the pharmaceutical composition is in a form in which the active ingredient layer is formed on the inert particle, the pharmaceutical composition may further include an additional layer between the inert particle and the active ingredient layer; and/or between the active ingredient layer and the release modifying agent-containing layer; and/or on the release modifying agent-containing layer. At this time, in case that the inert particle consists of or contains an organic acid and the additional layer is included between the inert particle and the active ingredient layer, the additional layer may function as an isolation layer for suppressing contact between the organic acid and the active ingredient. In this case, the contact between the active ingredient tegoprazan and the organic acid contained in the inert particle may be suppressed by the isolation layer, so that the stability of tegoprazan may be maintained at a high level, the storage stability thereof may be increased, and the effect of treating diseases mediated by acid pump antagonistic activity may be improved.

According to an example of the present disclosure, in the modified-release pharmaceutical composition of the present disclosure, an additional layer containing a pharmaceutically acceptable additive without the active ingredient may be positioned on a core, and a release modifying agent-containing layer may be formed on the additional layer. According to another example of the present disclosure, in the modified-release pharmaceutical composition of the present disclosure, an additional layer containing a pharmaceutically acceptable additive without the active ingredient may be positioned on an inert particle, an active ingredient layer may be positioned on the additional layer, and a release modifying agent-containing layer may be positioned on the active ingredient layer. According to still another example of the present disclosure, in the modified-release pharmaceutical composition of the present disclosure, an active ingredient layer may be formed on an inert particle, and an additional layer containing only a pharmaceutically acceptable additive without the active ingredient may be formed on the active ingredient layer. According to yet another example of the present disclosure, in the modified-release pharmaceutical composition of the present disclosure, an additional layer containing a pharmaceutically acceptable additive without the active ingredient may be formed on an inert particle, an active ingredient layer may be formed on the additional layer, an additional layer containing a pharmaceutically acceptable additive without the active ingredient may be formed on the active ingredient layer, and a release modifying agent-containing layer may be formed on the additional layer. In the pharmaceutical composition of the present disclosure, if necessary, an additional layer containing a pharmaceutically acceptable additive without the active ingredient may be formed on the release modifying agent layer of the above embodiments. In case that the inert particle contains an organic acid, an additional layer formed between the inert particle and the active ingredient layer may function as an isolation layer that suppresses the contact between the organic acid and the active ingredient.

The additional layer may be formed more appropriately or may not be formed, depending on the process and the type of material contained in each layer.

In examples of the present disclosure, the pharmaceutical composition may not include an additional layer, and the release modifying agent-containing layer in the pharmaceutical composition may function as an isolation layer.

In examples of the present disclosure, in case that a layer containing the organic acid is formed separately, an additional layer containing a pharmaceutically acceptable additive without the active ingredient may be positioned between the layer containing the organic acid and the layer containing the active ingredient and may function as an isolation layer that blocks the contact between the organic acid and the active ingredient. In examples of the present disclosure, the additional layer containing a pharmaceutically acceptable additive without the active ingredient may contain a polymer. The polymer may include at least one compound selected from the group consisting of methyl cellulose, ethyl cellulose, hydroxymethyl cellulose, methylhydroxyethyl cellulose, hydroxypropyl cellulose, hypromellose, polyvinyl pyrrolidone, and polyethylene glycol. Specifically, the polymer may include hypromellose. At this time, in case that the additional layer not containing the active ingredient contains hypromellose, the hypromellose may have a viscosity of 5 to 50 m·Pas, preferably 3 to 15 m·Pas, in an aqueous solution at 25° C.

In the present disclosure, the additional layer not containing the active ingredient may contain the polymer in an amount of about 30 to 99 wt %, specifically about 35 to 90 wt %, more specifically about 40 to 85 wt %, based on the total weight of the additional layers.

The additional layer not containing the active ingredient of the present disclosure may further contain a pharmaceutically acceptable additive in addition to the polymer. Examples of the additives that may be further contained include, but are not limited to, an anti-adhesion agent, a plasticizer, a surfactant, a disintegrant and an excipient, preferably, an anti-adhesion agent and/or a plasticizer. The content and type of the pharmaceutically acceptable additive may be appropriately selected by a person skilled in the art. For example, the pharmaceutically acceptable additive may be talc. A method for preparing the pharmaceutical composition according to the present disclosure may be performed according to a conventional process known in the pharmaceutical field. Coating according to this preparation method may be performed by a general coating method known in the art, and specifically, may be performed using a fluidized bed pellet coater. For example, in the modified-release pharmaceutical composition of the present disclosure, in case that the particle containing tegoprazan and/or the core is pellet and the release modifying agent layer (e.g., an enteric agent layer) is included on the core layer, the pharmaceutical composition of the present disclosure may be prepared by: i) spraying a coating solution, prepared by dissolving the active ingredient in any solvent, onto an inert particle, followed by drying to form an active ingredient layer; and ii) spraying a coating solution, prepared by dissolving a release modifying agent in any solvent, onto the active ingredient layer. This method is for illustrative purposes only, and the method of preparing the pharmaceutical composition is not limited thereto. In addition, in case that the pharmaceutical composition of the present disclosure includes a release modifying agent (for example, a sustained-release agent) inside the active ingredient layer, the coating solution prepared by dissolving the active ingredient contains the release modifying agent, and the active ingredient layer may be formed using the coating solution containing the release modifying agent. In addition, in case that the pharmaceutical composition of the present disclosure includes a plurality of release modifying agent layers, it may be prepared by performing the coating solution spraying and drying of step ii), and then spraying a coating solution, prepared by dissolving a release modifying agent (second) in any solvent, onto the release modifying agent (first) layer. In addition, the method of preparing the pharmaceutical composition of the present disclosure may further include a step of spraying and drying a coating solution for forming an isolation layer, before the coating solution spraying of step i) and/or ii). The isolation layer formed by coating before spraying the coating solution of the active ingredient layer and/or the modified-release layer may serve to spatially separate the layers from each other to prevent the contact between the components contained in the layers, thus increasing stability. In addition, the isolation layer may have an advantage in terms of ease of manufacture (yield, content, etc.) by roughening the surface during processing or making the surface clean during porous surface formation clean and enabling efficient formation of the layer to be coated subsequently, and may serve increase the abrasion resistance of the pharmaceutical composition.

In the present disclosure, the solvent of the coating solution may be selected from, for example, ethanol, purified water, isopropyl alcohol, acetone, and mixtures thereof, but is not limited thereto. The coating solution may contain pharmaceutically acceptable additives, including, but not limited to, a binding agent, a plasticizer, an anti-adhesion agent, a surfactant, a disintegrant, an excipient, or a mixture thereof.

In examples of the present disclosure, the modified-release pharmaceutical composition may be a capsule, a tablet, a pellet or a granule.

In examples of the present disclosure, in case that the pharmaceutical composition is a pellet, the pellet may include a core including an inert particle and an active ingredient-containing coating layer formed on the inert particle. The coating layer containing the active ingredient may contain a release modifying agent. In one embodiment, the pellet may be a pellet in which a release modifying agent-containing layer is formed on the core. The release modifying agent-containing layer may be one or more in number, and in case that the release modifying agent-containing layer has two or more layers, the release modifying agents contained in the adjacent layers may be different from each other. The release modifying agent contained in the active ingredient layer and the release modifying agent of the release modifying agent layer formed on the core may be each independently a sustained-release agent, an enteric agent, or both. In one embodiment, the release modifying agent contained in the active ingredient layer may be a sustained-release agent, and the release modifying agent of the release modifying agent-containing layer may be an enteric agent. In another embodiment, in case that the release modifying agent-containing layer includes two layers, the pharmaceutical composition may include the core; a first release modifying agent-containing layer formed on the core; and a second release modifying agent-containing layer formed on the first release modifying agent-containing layer, wherein the first release modifying agent may be a sustained-release agent, and the second release modifying agent may be an enteric agent. Wherein, an additional layer not containing the active ingredient may be included between the inert particle, the active ingredient-containing layer (active ingredient layer), the core, and the release modifying agent layer. Wherein, the active ingredient-containing layer and the release modifying agent-containing layer may contain a pharmaceutically acceptable additive. In this case, the inert particle, the active ingredient, the active ingredient-containing layer, the release modifying agent, the sustained-release agent, the enteric agent, the release modifying agent-containing layer, the additional layer not containing the active ingredient, and the pharmaceutically acceptable additive are the same as described above, unless there are contradictions.

In examples of the present disclosure, when the pharmaceutical composition is a granule, the granule may be a granule including: a core containing the active ingredient; and a release modifying agent-containing layer formed on the core. In this case, an additional layer not containing the active ingredient may be included between the core and the release modifying agent-containing layer. Alternatively, the granule may be a granule (wet granule or dry granule) formed from a mixture containing the active ingredient and a pharmaceutically acceptable additive. In this case, a release modifying agent-containing layer may be formed on the core which is the granule. At this time, an additional layer not containing the active ingredient may be included between the core and the release modifying agent-containing layer. Alternatively, the granule may be a granule (wet granule or dry granule) formed from a mixture containing the active ingredient, a pharmaceutically acceptable additive and a release modifying agent. At this time, a release modifying agent layer may be included on the core which is the granule. In the above-described granules, the release modifying agent-containing layer may be one or more in number, and in case that the release modifying agent-containing layer has two or more layers, the release modifying agents included in the layers may be different from each other. Wherein, the inert particle, the active ingredient, the active ingredient-containing layer, the release modifying agent, the release modifying agent-containing layer, the additional layer not containing the active ingredient, and the pharmaceutically acceptable additive are the same as described above, unless there are contradictions.

In examples of the present disclosure, in case that the pharmaceutical composition is a tablet, the tablet may be a tablet containing the active ingredient, and in this case, the tablet may be a core tablet by tableting a granule or pellet containing a pharmaceutically acceptable additive. Wherein, the core tablet may contain a release modifying agent. In addition, the tablet may be a tablet in which a release modifying agent-containing layer is formed on the core tablet. For example, the tablet may be formed by tableting a granule containing the active ingredient, wherein the granule may include either the core containing the active ingredient or a release modifying agent-containing layer positioned on the core. In this case, the core containing the active ingredient may be a granule (wet granule or dry granule) formed from a mixture containing the active ingredient and a pharmaceutically acceptable additive, and may further contain a release modifying agent. Alternatively, the tablet may be a tablet formed by tableting a granule (wet granule or dry granule) formed from a mixture containing the active ingredient, a pharmaceutically acceptable additive, and a release modifying agent. The tablet may further include a release modifying agent on the tablet. In this case, an additional layer containing a pharmaceutically acceptable additive without the active ingredient may be further included between the core and the release modifying agent-containing layer. Wherein, the granule, the pellet, the active ingredient, the layer containing the active ingredient, the release modifying agent, the release modifying agent-containing layer, the additional layer not containing the active ingredient, and the pharmaceutically acceptable additive are the same as described above, unless there are contradictions.

The modified-release pharmaceutical composition according to the present disclosure may be formulated as an oral dosage form.

In the present disclosure, the modified-release composition may be formulated as a capsule. In this case, the capsule may be filled with a tablet, a granule, a pellet, or a mixture thereof, in which the tablet, the granule and the pellet are as described above. In this case, the capsule may further contain a powder which is the active ingredient itself or a powder which is either a mixture of the active ingredient and a pharmaceutically acceptable additive or a mixture of the active ingredient, a pharmaceutically acceptable additive and a release modifying agent. For example, the capsule may be filled with the pellet, the tablet or the granule, or filled with a mixture of the powder and the pellet, a mixture of the powder and the granule, a mixture of the powder and the tablet, a mixture of the pellet and the tablet, a mixture of the pellet and the granule, or a mixture of the tablet and the granule, or may be filled with a mixture of the powder, the pellet and the tablet, a mixture of the powder, the pellet and the granule, a mixture of the powder, the granule and the pellet, a mixture of the pellet, the tablet and the granule, or a mixture of the powder, the pellet, the tablet and the granule.

According to examples of the present disclosure, the capsule may be filled with the pellet. For example, the capsule may be a capsule filled with a pellet that includes an inert particle and an active ingredient-containing coating layer, which contains the active ingredient, formed on the inert particle. Here, the active ingredient-containing coating layer includes a release modifying agent, and/or a release modifying agent-containing layer is formed on the core.

In the present disclosure, the modified-release composition may be formulated as a tablet. The tablet may be a tablet including: a core including an inert particle and an active ingredient-containing coating layer formed on the inert particle; and a release modifying agent-containing layer formed on the core, in which the inert particle may be a core tablet formed by tableting a granule containing a pharmaceutically acceptable additive. Alternatively, the tablet may be formed by tableting a granule, a pellet, or a mixture thereof, in which the granule and the pellet are as described above. Wherein, the tablet may further contain a powder that is the active ingredient itself. For example, the tablet may be formed by tableting a granule containing the active ingredient, wherein the granule may include a core containing the active ingredient, which includes a release modifying agent on the core, and/or a release modifying agent-containing layer positioned on the core. In this case, the core containing the active ingredient may be a granule (wet granule or dry granule) formed from a mixture containing the active ingredient and a pharmaceutically acceptable additive, and in case that the core includes a release modifying agent, the mixture may further contain the release modifying agent. Alternatively, the tablet may be a tablet formed by tableting a granule (wet granule or dry granule) formed from a mixture containing the active ingredient, a pharmaceutically acceptable additive and a release modifying agent.

In case that the modified-release pharmaceutical composition of the present disclosure includes both a sustained-release agent and an enteric agent, the modified-release pharmaceutical composition of the present disclosure may be present in a form in which a sustained-release portion including the active ingredient and a sustained-release agent and an enteric portion including the active ingredient and an enteric agent are separated from each other. For example, in case that the pharmaceutical composition is a capsule, the capsule includes: a sustained-release portion including the active ingredient and the sustained-release agent; and the enteric portion including the active ingredient and an enteric agent, wherein the sustained-release portion and the enteric portion may each independently be a powder, a granule, a pellet, or a tablet, and the capsule may be filled with the sustained-release portion and the enteric portion, which are each independently a powder, a granule, a pellet, or a tablet. Alternatively, in the pharmaceutical composition of the present disclosure, the active ingredient, the sustained-release agent and the enteric agent may be present in the form of a single particle (granule, pellet, or tablet).

The modified-release pharmaceutical composition of the present disclosure may further contain a pharmaceutically acceptable additive. Pharmaceutically acceptable additives of the present disclosure may include, but are not limited to, a binding agent, an anti-adhesion agent, a plasticizer, a surfactant, a stabilizer, a disintegrant, and an excipient. One or more of the additives may be included in the active ingredient layer, and the content and type thereof may be appropriately selected by a person skilled in the art within a range that does not affect the stability or effect of the active ingredient. The binding agent may be, for example, polyvinyl alcohol, ethyl cellulose, a polyethylene glycol-polyvinyl alcohol copolymer, hydroxypropyl cellulose, hypromellose (hydroxypropyl methyl cellulose), microcrystalline cellulose, mannitol, sucrose, lactose, polyethylene glycol, polyvinylpyrrolidone, sodium carboxymethylcellulose, pregelatinized starch, natural gum, synthetic gum, a polyvinylpyrrolidone copolymer, povidone, gelatin, starch, or highly dispersible silica, but is limited thereto. The anti-adhesion agent may be, for example, light anhydrous silicic acid, hydrated silicon dioxide, talc, or stearic acid, but is not limited thereto. The plasticizer may be, for example, acetyl triethyl citrate, triethyl citrate (citric acid triethyl), diethyl phthalate, polyethylene glycol, or triacetin, but is not limited thereto. However, hydrophilic and highly reactive plasticizers such as polyethylene glycol may affect long-term stability, and thus may not be added depending on the purpose. The surfactant may be, for example, sodium lauryl sulfate, polyethylene, glycol, poloxamer, or polysorbate (polysorbate 20, 40, 60 or 80), but is not limited thereto. The stabilizer may be, for example, sodium carbonate, sodium hydrogen carbonate, potassium carbonate, potassium hydrogen carbonate, magnesium carbonate, magnesium oxide, magnesium hydroxide, magnesium aluminometasilicate, magnesium silicate, magnesium aluminate, synthetic hydrotalcite, or aluminum magnesium hydroxide, but is not limited thereto. Examples of the disintegrant include, but are not limited to, sodium starch glycolate, corn starch, potato starch, pregelatinized starch, algins such as sodium alginate or alginic acid, celluloses such as microcrystalline cellulose, hydroxypropyl cellulose or carboxymethyl cellulose, crosslinked celluloses such as carmellose or croscarmellose sodium, gums such as guar gum or xanthan gum, and effervescent agents such as sodium bicarbonate or citric acid.

The modified-release pharmaceutical composition of the present disclosure may be used for the prevention or treatment of diseases mediated by acid pump antagonistic activity.

The present disclosure provides the modified-release pharmaceutical composition of the present disclosure for the prevention or treatment of a disease mediated by acid pump antagonistic activity.

The diseases mediated by acid pump antagonistic activity may be, but is not limited to, gastrointestinal tract disease, gastroesophageal disease, gastroesophageal reflux disease (GERD), peptic ulcer, gastric ulcer, duodenal ulcer, NSAID-induced ulcer, gastritis, Helicobacter pylori infectious disease, indigestion, functional indigestion, Zollinger-Ellison syndrome, non-erosive reflux disease (NERD), visceral-related pain, heartburn, nausea, esophagitis, difficulty swallowing, drooling, airway disorder, or asthma, preferably gastroesophageal reflux disease (GERD).

The “gastroesophageal reflux disease (GERD)” refers to a condition in which the stomach contents are refluxed into the esophagus to cause symptoms causing discomfort in daily life or cause complications. The gastroesophageal reflux disease (GERD) may be classified into erosive esophagitis (EE) and non-erosive reflux disease (NERD).

In the present specification, “prevention” includes preventing, delaying, or inhibiting the development of a disease, and “treatment” includes alleviating disease symptoms, or preventing disease worsening, or delaying, or inhibiting a disease.

The modified-release pharmaceutical composition of the present disclosure can modify the release of the active ingredient tegoprazan, thus enabling the tegoprazan to be maintained at a high concentration in the blood until a certain time after taking the same. Thus, the modified-release pharmaceutical composition may exhibit an excellent therapeutic effect for the above diseases for a long period of time, and may significantly improve the patient's medication compliance. In addition, the pharmaceutical composition may exhibit excellent storage stability when formulated, and also show excellent dissolution even in an environment having a pH higher than that of a gastric juice environment, and as a result, exhibit excellent dissolution under an intestinal fluid environment, for example, a duodenal environment. In addition, the pharmaceutical composition may achieve a therapeutically excellent blood concentration of tegoprazan through dissolution modifying without lowering the dissolution rate of tegoprazan even when tegoprazan is released in a delayed manner, and the composition may achieve excellent sustained release of tegoprazan even in the intestinal fluid environment.

The present disclosure provides a use of the modified-release pharmaceutical composition of the present disclosure for the prevention or treatment of diseases mediated by acid pump antagonistic activity.

The present disclosure provides a use of the modified-release pharmaceutical composition of the present disclosure in the manufacture of a medicament for the prevention or treatment of diseases mediated by acid pump antagonistic activity.

The present disclosure provides a method for preventing or treating diseases mediated by acid pump antagonistic activity, the method including administering an effective amount of the modified-release pharmaceutical composition of the present disclosure to a subject in need thereof.

In the present disclosure, the term “subject” refers to mammals, including, but not limited to, humans, guinea pigs, monkeys, cows, horses, sheep, pigs, chickens, turkeys, quails, cats, dogs, mice, mice, or rabbits. Specifically, the subject may be a human being.

The modified-release pharmaceutical composition in the above-described use and the prevention or treatment method is the same as described above, unless there are contradictions.

The modified-release pharmaceutical composition of the present disclosure may be used in combination with a pharmaceutical composition for immediate release of the active ingredient.

In the present disclosure, the term “effective amount” as used herein refers to an amount sufficient to treat a disease at a reasonable benefit/risk ratio applicable to medical treatment, and the level of the effective amount may be determined by factors, including the type of disease, the severity of disease, drug activity, drug sensitivity, the time of administration, the route of administration and the rate of excretion, the duration of treatment and drug used concurrently, and other factors well known in the medical arts. It is important to administer an amount at which a maximum effect can be obtained with a minimum amount while not causing a side effect in consideration of all the factors, and such an amount can be determined by a person of ordinary skill in the art.

Formulation

The present disclosure provides a formulation including: a modified-release first pharmaceutical composition which contains, tegoprazan, an optical isomer thereof, a pharmaceutically acceptable salt thereof, a hydrate or solvate thereof, or a mixture thereof as an active ingredient; and a second pharmaceutical composition which contains tegoprazan, an optical isomer thereof, a pharmaceutically acceptable salt thereof, a hydrate or solvate thereof, or a mixture thereof as an active ingredient, and releases the active ingredient immediately.

According to the present disclosure, immediate release and modified-release of tegoprazan from the formulation may be achieved, a rapid drug effect may be obtained, and at the same time, tegoprazan may be maintained at a high concentration in the blood until a certain time after taking the formulation. Thus, the formulation of the present disclosure may have excellent therapeutic effect and may significantly improve the patient's medication compliance. The formulation of the present disclosure may be in a form such that the active ingredient is released within a short time after taking the formulation and then additional release of the active ingredient occurs after a certain period of time. In this case, the blood concentration of tegoprazan may increases immediately after taking the formulation, and then the blood concentration of tegoprazan may increase once again after a certain period of time (or at a specific location or under a specific condition). For example, after administration of the drug, tegoprazan may be rapidly released immediately in the gastric juice environment, so that the concentration of tegoprazan in the blood may increase rapidly, and after a certain period of time, the concentration of tegoprazan in the blood may increase once again while tegoprazan is dissoluted from the formulation in an intestinal fluid environment such as the duodenum, for example, at pH 5 or higher. That is, when tegoprazan is dissoluteb from the formulation in a continuous or pulsatile manner after administration of the formulation, the blood concentration of tegoprazan may increase over two times in a continuous or pulsatile manner. Alternatively, the formulation of the present disclosure may be configured such that the active ingredient is released within a short time after administration, and then the active ingredient is released in a sustained manner for a certain period of time. In this case, the blood concentration of tegoprazan may increase immediately after taking the formulation, and the release of tegoprazan may be sustained, so that the blood concentration of tegoprazan may also be maintained or increased. Alternatively, the formulation of the present disclosure may be in a form such that the active ingredient is released within a short time after administration, and after a certain period of time (or at a specific location or in a specific condition), additional release of the active ingredient begins to occur, and then the release is sustained for a certain period of time. In this case, after taking the formulation, the blood concentration of tegoprazan may increase immediately, and after a certain period of time, the blood concentration of tegoprazan may increase once again, and then the release of tegoprazan may be sustained, so that the blood concentration of the tegoprazan may be maintained or increased. For example, after drug administration, the concentration of tegoprazan may be rapidly released immediately in the gastric juice environment, so that the concentration of tegoprazan in the blood rapidly increase, and after a certain period of time, the concentration of tegoprazan in the blood may increase once again while tegoprazan is dissoluted from the formulation in an intestinal fluid environment such as the duodenum, for example, at pH 5 or higher, and then tegoprazan may be sustainedly dissoluted from the formulation for a certain time, so that the blood concentration of tegoprazan may be maintained or increased. Therefore, administration of the formulation of the present disclosure may exhibit a sustained pharmacological effect with a rapid drug effect even when the dose of tegoprazan in the formulation is low.

In the formulation containing the modified-release first pharmaceutical composition of the present disclosure, the contents described above in the preceding “modified-release pharmaceutical composition” section may be equally applied, unless there are contradictions. Thus, the above-described structure of the “modified-release pharmaceutical composition”, the above-described inert particle, core, release modifying agent, release modifying agent-containing layer, sustained-release agent, sustained-release agent-containing layer, enteric agent, enteric agent-containing layer, pellet, tablet, granule, capsule, pharmaceutically acceptable additive, components included in the composition, and the content ratio of the components may likewise be applied to the modified-release first pharmaceutical composition, unless there are contradictions.

In examples of the present disclosure, the second pharmaceutical composition from which the active ingredient is immediately released may be appropriately formulated so that the active ingredient may be immediately released. In one example, the second pharmaceutical composition may include an inert particle and an active ingredient layer, which contains an active ingredient, positioned on the inert particle. In this case, an additional layer not containing the active ingredient but containing only a pharmaceutically acceptable additive may be included between the inert particle and the active ingredient layer, but is not limited thereto, and the additional layer may not be included. In case that the additional layer is included, the additional layer may function to facilitate the formation of a subsequent layer. The additional layer may be positioned between the inert particle and the active ingredient layer and/or on the active ingredient layer. The second pharmaceutical composition including the inert particle and the active ingredient layer may be a pellet. In another example, the second pharmaceutical composition may be a particle prepared from a mixture containing a pharmaceutically acceptable additive and the active ingredient. For example, the second pharmaceutical composition may be a granule formed from a mixture containing the active ingredient and a pharmaceutically acceptable additive. Alternatively, the second pharmaceutical composition may be a mixture of a pharmaceutically acceptable additive and the active ingredient, wherein the mixture may be in a powder form. In yet another example, the second pharmaceutical composition may also be a tablet prepared from the pellet, the granule, the powder, or a mixture thereof. Wherein, the inert particle, the granule, the pellet, the powder, the tablet, the additional layer, function of the additional layer, the component and content of the pharmaceutically acceptable additive are as described above with respect to the “modified release pharmaceutical composition”, unless there are contradictions.

In examples of the present disclosure, the first pharmaceutical composition and the second pharmaceutical composition may be separated from each other and exist as separate particles.

In examples of the present disclosure, the first pharmaceutical composition and the second pharmaceutical composition may be each independently a powder, a pellet, a granule or a tablet.

In the examples of the present disclosure, the first pharmaceutical composition may be a pellet, a granule or a tablet, wherein the pellet, granule or tablet is as described above with respect to the “modified release pharmaceutical composition”.

In examples of the present disclosure, the second pharmaceutical composition may be a powder, a pellet, a granule or a tablet. In one example, the second pharmaceutical composition may be a powder, and in this case, the second pharmaceutical composition may be a mixture of the active ingredient and a pharmaceutically acceptable additive. In another example, the second pharmaceutical composition may be a pellet. In this case, the pellet may include an inert particle and a coating layer, which contains the active ingredient positioned on the inert particle. The pellet may further include an additional layer, which contains only a pharmaceutically acceptable additive without the active ingredient, wherein the additional layer may be positioned between the inert particle and the active ingredient layer and/or on the active ingredient layer. Wherein, the inert particle, the active ingredient, the layer containing the active ingredient, and the additional layer not containing the active ingredient but containing only a pharmaceutically acceptable additive are as described above with respect to the “modified-release pharmaceutical composition”, unless there are contradictions.

In another example, the second pharmaceutical composition may be a granule, and the granule may include an inert particle and a coating layer containing the active ingredient on the inert particle. In addition, the granule may be a granule (wet granule or dry granule) formed from a mixture containing the active ingredient and a pharmaceutically acceptable additive. Wherein, the inert particle, the active ingredient, the pharmaceutically acceptable additive, etc. are as described above with respect to the “modified-release pharmaceutical composition”, unless there are contradictions.

In another example, the second pharmaceutical composition may be a tablet, and the tablet may include an inert particle and an active ingredient layer positioned on the inert particle. Wherein, the inert particle may be a core tablet formed by tableting a granule or pellet containing a pharmaceutically acceptable additive. Alternatively, the tablet may be formed by tableting a granule containing the active ingredient, wherein the granule may be a granule (wet granule or dry granule) formed from a mixture containing the active ingredient and a pharmaceutically acceptable additive. In this case, an additional layer consisting of only pharmaceutically acceptable additives without the active ingredient may be included between the inert particle and the active ingredient-containing layer and/or on the active ingredient-containing layer. Wherein, the inert particle, the active ingredient, the active ingredient-containing layer, the additional layer not containing the active ingredient, etc. are as described above with respect to the “modified-release pharmaceutical composition”, unless there are contradictions.

In the examples of the present disclosure, the formulation may be formulated as an oral dosage form.

In case that the first pharmaceutical composition and the second pharmaceutical composition are separated from each other and present as separate particles, the first pharmaceutical composition and the second pharmaceutical composition may be formulated as a single unit dosage form or the respective unit dosage forms.

In case that the first pharmaceutical composition and the second pharmaceutical composition are formulated as the respective unit dosage forms, the unit dosage form of the first pharmaceutical composition and the unit dosage form of the second pharmaceutical composition may be administered simultaneously. The unit dosage form of the first pharmaceutical composition may be a powder, a pellet, a granule or a tablet, or may be a capsule filled with the pellet, granule or tablet, and the unit dosage form of the second pharmaceutical composition may be a powder, a pellet, a granule or a tablet, may be a capsule filled with the powder, pellet, granule or tablet, and the unit dosage form of the first pharmaceutical composition and the unit dosage form of the second pharmaceutical composition may be independent of each other. Wherein, the powder, the pellet, the granule, the tablet and the capsule may be the same as described above, unless there are contradictions.

The first pharmaceutical composition and the second pharmaceutical composition may be formulated as a single unit dosage form.

In examples of the present disclosure, the formulation may be formulated as a capsule.

In examples of the present disclosure, in case that the first pharmaceutical composition and the second pharmaceutical composition are in the form of separate particles, the formulation may be a capsule filled with the first pharmaceutical composition and the second pharmaceutical composition. In this case, the first pharmaceutical composition and the second pharmaceutical composition may be each independently a powder, a pellet, a granule or a tablet, and may be in the same form or different forms. Wherein, the powder, the pellet, the granule or the tablet is as described above. For example, the formulation may be a capsule filled with the modified-release first pharmaceutical composition which is in the form of a pellet and the second pharmaceutical composition which is in the form of a pellet and from which the active ingredient is immediately released. Alternatively, the formulation may be a capsule filled with the modified-release first pharmaceutical composition which is in the form of a pellet and the second pharmaceutical composition which is in the form of a tablet or granule and from which the active ingredient is immediately released. Wherein, the first pharmaceutical composition which is the pellet, granule or tablet is as described above with respect to the “modified-release pharmaceutical composition”, and the second pharmaceutical composition which is the powder, pellet, granule or tablet is as described above.

In examples of the present disclosure, in case that the formulation is a capsule, the first pharmaceutical composition may be a pellet, and the second pharmaceutical composition may be a powder, a granule or a tablet.

In one embodiment, the first pharmaceutical composition, which is a pellet, may include: an inert particle; an active ingredient layer, which contains the active ingredient, formed on the inert particle; and a release modifying agent (containing) layer, which contains a release modifying agent, formed on the active ingredient layer. The release modifying agent may be at least one selected from the group consisting of a sustained-release agent and an enteric agent.

In another embodiment, the first pharmaceutical composition, which is a pellet, may include: an active ingredient layer, which contains the active ingredient and a first release modifying agent, formed on the inert particle; and a second release modifying agent (containing) layer, which contains a second release modifying agent, formed on the active ingredient layer. The first and second release modifying agents may be each independently at least one selected from the group consisting of a sustained-release agent and an enteric agent. For example, the first release modifying agent may be a sustained-release agent, and the second release modifying agent may be an enteric agent.

In still another embodiment, the first pharmaceutical composition, which is a pellet, may include: an active ingredient layer, which contains the active ingredient and a first release modifying agent, formed on the inert particle; a second release modifying agent (containing) layer, which contains a second release modifying agent, formed on the active ingredient layer; and a third release modifying agent (containing) layer, which contains a third release modifying agent, formed on the second release modifying agent (containing) layer. The first, second and third release modifying agents may be each independently at least one selected from the group consisting of a sustained-release agent and an enteric agent. For example, the first release modifying agent and the second release modifying agent may be sustained-release agents, and the third release modifying agent may be an enteric agent, the first release modifying agent and the second release modifying agent may be the same as or different from each other.

In one embodiment, the second pharmaceutical composition, which is a powder, may be a powdery mixture which is a mixture of the active ingredient and a pharmaceutically acceptable additive. In this case, the powdery mixture may be distinguished from a granule. In another embodiment, the second pharmaceutical composition, which is a granule, may be a granule prepared by a granulation process from a mixture containing the active ingredient and a pharmaceutically acceptable additive.

Wherein, the pellet, the inert particle, the release modifying agent, the sustained-release agent, the enteric agent, the granule, the powder, and the pharmaceutically acceptable additive are as described above.

In examples of the present disclosure, the formulation may be formulated as a tablet.

In examples of the present disclosure, in case that the first pharmaceutical composition and the second pharmaceutical composition are in the form of separate particles, the formulation may be a tablet including the first pharmaceutical composition and the second pharmaceutical composition. In one embodiment, the tablet may be a multilayered-tablet including a first layer containing a first pharmaceutical composition and a second layer containing the second pharmaceutical composition, wherein the first layer may release the active ingredient in a modified manner (e.g., a delayed manner, a sustained manner, or both), and the second layer may release the active ingredient immediately. Here, the tablet may be prepared by tableting the first pharmaceutical composition which is in the form of a granule or a pellet and the second pharmaceutical composition which is in the form of powder, granule or pellet. In another embodiment, the tablet may be a multilayered-tablet in form of tab-in-tab including a first layer, which is a core tablet including the first pharmaceutical composition, and a second layer, which surrounds the first layer and includes the second pharmaceutical composition. In this case, the tablet may be prepared by tableting the first pharmaceutical composition which is in the form of a granule or a pellet to prepare a core tablet, and tableting the core tablet and the second pharmaceutical composition which is in the form of powder, granule or pellet. Wherein, the first pharmaceutical composition, which is a pellet or a granule, is as described above with respect to the “modified-released pharmaceutical composition”, and the second pharmaceutical composition, which is a powder, a pellet or a granule, is as described above.

In case that the first pharmaceutical composition, which is a pellet or a granule, contains an enteric agent, the formulation of the present disclosure, which is a tablet prepared from the pellet or granule, may release the active ingredient from the first layer in a delayed manner. In case that the first pharmaceutical composition, which is a pellet or a granule, contains a sustained-release agent, the formulation of the present disclosure, which is a tablet prepared from the pellet or granule, may release the active ingredient from the first layer in a sustained manner. In case that the first pharmaceutical composition, which is a pellet or granule, contains an enteric agent and a sustained-release agent, the formulation of the present disclosure, which is a tablet prepared from the pellet or granule, may release the active ingredient from the first layer in a delayed manner and in a sustained manner.

In examples of the present disclosure, the first pharmaceutical composition and the second pharmaceutical composition may be present together in a single particle.

In the examples of the present disclosure, the first pharmaceutical composition is as described above with respect to the “modified-release pharmaceutical composition”, and the second pharmaceutical composition may be positioned on the first pharmaceutical composition.

In the examples of the present disclosure, the first pharmaceutical composition includes a core containing the active ingredient, and includes a release modifying agent on the core and/or includes a release modifying agent layer, which contains a release modifying agent, positioned on the core, wherein the second pharmaceutical composition may be positioned on the core or on the release modifying agent layer formed on the core. In this case, the formulation including the first pharmaceutical composition and the second pharmaceutical composition may further include an additional layer composed of only a pharmaceutically acceptable additive without containing the active ingredient. The function of the additional layer and the substance included therein are as described above with respect to the “modified-release pharmaceutical composition”. When the above-described formulation is administered, the active ingredient in the second pharmaceutical composition on the outside of the formulation may be dissoluted in the gastric juice environment, and the release modifying agent is dissolved from the first pharmaceutical composition, the active ingredient may be dissoluted in a region ranging from the gastric juice environment to the intestinal environment, or the active ingredient may be dissoluted in the intestinal environment (e.g., duodenum, etc.) which is not the gastric juice environment, or may be dissoluted in the intestinal environment, which is not the gastric juice environment, and may be dissoluted in a sustained manner over a certain time. In examples of the present disclosure, in case that the first pharmaceutical composition and the second pharmaceutical composition are present together in a single particle, the particle may be a granule, a pellet or a tablet.

In the examples of the present disclosure, in case that the particle is a pellet, the pellet may include: a core including, an inert particle and a coating layer, which contains the active ingredient and a sustained-release agent, formed on the inert layer; an enteric agent-containing layer on the core; and an active ingredient layer, which contains the active ingredient, positioned on the enteric agent-containing layer. In this case, an additional layer not containing the active ingredient may be included between the inert particle, the coating layer including the active ingredient, the core, and the enteric agent-containing layer. In case that an additional layer is included between the core and the enteric agent-containing layer, the additional layer may be a sustained-release agent-containing layer including a sustained-release agent. Wherein, the inert particle, the active ingredient, the coating layer containing the active ingredient, the sustained-release agent, the enteric agent, the enteric agent-containing layer, the sustained-release agent-containing layer, and the additional layer not containing the active ingredient are as described above with respect to the “modified-release pharmaceutical composition”, unless there are contradictions. In the examples of the present disclosure, in case that the particle is a granule, the granule may include: a core including, an inert particle and a coating layer, which contains the active ingredient and a sustained-release agent, formed on the inert particle; an enteric agent-containing layer on the core; and an active ingredient layer, which contains the active ingredient, positioned on the enteric agent-containing layer. Alternatively, the granule may include: a core which is a granule (wet granule or dry granule) formed from a mixture containing the active ingredient, a sustained-release agent and a pharmaceutically acceptable additive; an enteric agent-containing layer positioned on the core; and an active ingredient layer, which contains the active ingredient, positioned on the enteric agent-containing layer. Alternatively, the granule may include: a granule (wet granule or dry granule) formed from a mixture containing at least one of the active ingredient, a pharmaceutically acceptable additive, a sustained-release agent and an enteric agent; and an active ingredient layer, which contains the active ingredient, positioned on the granule. In this case, an additional layer not containing an active ingredient may be included between the inert particle, the layer containing the active ingredient, the core, and the layer containing the enteric agent. In case that an additional layer is included between the core and the enteric agent-containing layer, the additional layer may be a sustained-release agent-containing layer containing a sustained-release agent. Wherein, the inert particle, the active ingredient, the layer containing the active ingredient, the sustained-release agent, the enteric agent, the enteric agent-containing layer, the sustained-release agent-containing layer, and the additional layer not containing the active ingredient are as described above with respect to the “modified-release pharmaceutical composition”, unless there are contradictions.

In examples of the present disclosure, in case that the particle is a tablet, the tablet may include: a core including, an inert particle; an active ingredient layer, which contains an active ingredient and a sustained-release agent, formed on the inert particle; an enteric agent-containing layer positioned on the core; and an active ingredient layer positioned on the enteric agent-containing layer containing the active ingredient. In this case, the inert particle may be a core tablet formed by tableting a granule or pellet containing a pharmaceutically acceptable additive. Alternatively, the tablet may include: a core tablet formed by tableting a granule containing the active ingredient and a sustained-release agent; an enteric agent-containing layer positioned on the core tablet; and an active ingredient layer, which contains the active ingredient, positioned on the enteric agent-containing layer. In this case, the granule may be a granule (wet granule or dry granule) prepared from a mixture of the active ingredient and a pharmaceutically acceptable additive. Alternatively, the tablet may include: a core tablet formed by tableting a granule (wet granule or dry granule) formed from a mixture containing the active ingredient, a pharmaceutically acceptable additive and an enteric agent; and an active ingredient layer, which contains the active ingredient, positioned on the core tablet. Here, an additional layer composed of a pharmaceutically acceptable additive without the active ingredient may be further included between the inert particle, the layer containing the active ingredient, the core, and the layer containing the enteric agent. In case that an additional layer is included between the core (core tablet) and the enteric agent-containing layer, the additional layer may be a sustained-release agent-containing layer containing a sustained-release agent. Here, the inert particle, the active ingredient, the layer containing the active ingredient, the sustained-release agent, the enteric agent, the enteric agent-containing layer, the sustained-release agent-containing layer, and the additional layer not containing the active ingredient are as described above with respect to the “modified-release pharmaceutical composition”, unless there are contradictions. In the present disclosure, the formulation may be a capsule. In this case, the capsule may be filled with a powder, a tablet, a granule, a pellet, or a mixture thereof, in which the powder, tablet, granule and pellet are as described above. For example, the capsule may be filled with each of a powder, a pellet, a tablet or a granule, or filled with a mixture of two or three or more selected from a powder, a tablet, a pellet and a granule, or may be filled with a mixture of a powder, a pellet, a tablet and a granule.

According to examples of the present disclosure, the capsule may be filled with a pellet. For example, the capsule may be a capsule filled with a pellet including: a core including, an inert particle and a coating layer containing the active ingredient and a sustained-release agent formed on the inert particle; an enteric agent-containing layer on the core; and a layer, which contains the active ingredient, positioned on the enteric agent-containing layer.

In the present disclosure, the formulation may be a tablet. The tablet may be prepared by tableting a powder, a granule, a pellet, or a mixture thereof, in which the powder, the pellet or the granule is as described above unless there are contradictions. For example, the tablet may be a multilayered-tablet including: a core including an inert particle and a coating layer, which contains the active ingredient and a sustained-release agent, formed on the inert particle; an enteric agent-containing layer on the core; and an active ingredient layer, which contains the active ingredient, positioned on the enteric agent-containing layer. In this case, the inert particle may be a core tablet formed by tableting a granule containing a pharmaceutically acceptable additive. Alternatively, the tablet may include: a core tablet formed by tableting a granule containing the active ingredient and a sustained-release agent; an enteric agent-containing layer positioned on the core tablet; and an active ingredient layer, which contains the active ingredient, positioned on the enteric agent-containing layer. In this case, the granule may be a granule (wet granule or dry granule) prepared from a mixture of the active ingredient, a sustained-release agent and a pharmaceutically acceptable additive. Alternatively, the tablet may be a tablet formed by tableting a granule (wet granule or dry granule) formed from a mixture containing the active ingredient, a pharmaceutically acceptable additive and an enteric agent.

In the examples of the present disclosure, the weight ratio between the active ingredients included in the first pharmaceutical composition and the second pharmaceutical composition may be about 5:1 to 1:5 as tegoprazan (free base form) (w:w), specifically about 3:1 to 1:3 (w:w). In examples of the present disclosure, the weight ratio between tegoprazan in the first pharmaceutical composition and tegoprazan in the second pharmaceutical composition may be 2:1 to 1:2. In examples of the present disclosure, the weight ratio between tegoprazan in the first pharmaceutical composition and tegoprazan in the second pharmaceutical composition may be 2:1 to 1:1.

In the examples of the present disclosure, the formulation may contain tegoprazan (free base form) as an active ingredient in an amount of 10 mg to 200 mg, specifically 15 mg to 150 mg, per unit dosage form. For example, the modified-release first pharmaceutical composition in the formulation may contain tegoprazan (free base form) as an active ingredient in an amount of about 5 mg to 100 mg per unit dosage form, and the second pharmaceutical composition that releases the active ingredient immediately may contain tegoprazan (free base form) as an active ingredient in an amount of about 5 mg to 100 mg per unit dosage form.

In the formulation of the present disclosure, the first pharmaceutical composition and the second pharmaceutical composition may be included in the formulation in an appropriate ratio range. According to an embodiment of the present disclosure, the weight ratio of the first pharmaceutical composition to the second pharmaceutical composition included in the formulation may be about 10:1 to 1:10, preferably about 7:1 to 1:7, more preferably about 5:1 to 1:5. In examples of the present disclosure, the weight ratio of the first pharmaceutical composition to the second pharmaceutical composition may be 3:1 to 1:3, or 2:1 to 1:2. In the present disclosure, the formulation may further include a pharmaceutically acceptable additive. The pharmaceutically acceptable additive may be included in the first pharmaceutical composition, or the second pharmaceutical composition, or the first and second pharmaceutical compositions, and may be included outside the first pharmaceutical composition and the second pharmaceutical composition. Example of the additive may include, but is not limited to a binding agent, an anti-adhesion agent, a plasticizer, a surfactant, a stabilizer, a disintegrant, and an excipient. One or more of the additives may be included in the active ingredient layer, and the content and type thereof may be appropriately selected by a person skilled in the art within a range that does not affect the stability or effect of the active ingredient. The additive included in the first pharmaceutical composition and the additive included in the second pharmaceutical composition may be the same as or different from each other. The binding agent may be, but is not limited to, for example, polyvinyl alcohol, ethyl cellulose, a polyethylene glycol-polyvinyl alcohol copolymer, hydroxypropyl cellulose, hypromellose (hydroxypropyl methyl cellulose), microcrystalline cellulose, mannitol, sucrose, lactose, polyethylene glycol, polyvinylpyrrolidone, sodium carboxymethylcellulose, pregelatinized starch, natural gum, synthetic gum, a polyvinylpyrrolidone copolymer, povidone, gelatin, starch, or highly dispersible silica. The anti-adhesion agent may be, but is not limited to, for example, light anhydrous silicic acid, hydrated silicon dioxide, talc, or stearic acid. The plasticizer may be, but is not limited to, for example, acetyl triethyl citrate, triethyl citrate, diethyl phthalate, polyethylene glycol, or triacetin. However, hydrophilic and highly reactive plasticizers such as polyethylene glycol may affect long-term stability, and thus may not be added depending on the purpose. The surfactant may be, for example, sodium lauryl sulfate, polyethylene, glycol, poloxamer, or polysorbate (polysorbate 20, 40, 60 or 80), but is not limited thereto. The stabilizer may be, for example, sodium carbonate, sodium hydrogen carbonate, potassium carbonate, potassium hydrogen carbonate, magnesium carbonate, magnesium oxide, magnesium hydroxide, magnesium aluminometasilicate, magnesium silicate, magnesium aluminate, synthetic hydrotalcite, or aluminum magnesium hydroxide, but is not limited thereto. Examples of the disintegrant include, but are not limited to, sodium starch glycolate, corn starch, potato starch, pregelatinized starch, algins such as sodium alginate or alginic acid, celluloses such as microcrystalline cellulose, hydroxypropyl cellulose or carboxymethyl cellulose, crosslinked celluloses such as carmellose or croscarmellose sodium, gums such as guar gum or xanthan gum, and effervescent agents such as sodium bicarbonate or citric acid.

In the present disclosure, the formulation including the first pharmaceutical composition and the second pharmaceutical composition may be used for the prevention or treatment of diseases mediated by acid pump antagonistic activity.

The present disclosure provides the formulation of the present disclosure including the first pharmaceutical composition and the second pharmaceutical composition for the prevention or treatment of diseases mediated by acid pump antagonistic activity.

The formulation including the first pharmaceutical composition and the second pharmaceutical composition according to the present disclosure may be effectively for the treatment of diseases mediated by acid pump antagonistic activity, including, but not limited to, gastrointestinal tract disease, gastroesophageal disease, gastroesophageal reflux disease (GERD), peptic ulcer, gastric ulcer, duodenal ulcer, NSAID-induced ulcer, gastritis, Helicobacter pylori infectious disease, indigestion, functional indigestion, Zollinger-Ellison syndrome, non-erosive reflux disease (NERD), visceral-related pain, heartburn, nausea, esophagitis, difficulty swallowing, drooling, airway disorder, or asthma.

Since the formulation including the first pharmaceutical composition and the second pharmaceutical composition according to the present disclosure include an immediate-release portion and a modified-release portion, it may exhibit a drug effect rapidly, and at the same time, enables tegoprazan to be maintained at a high concentration in the blood until a certain time after taking the formulation. Thus, the formulation may exhibit an excellent therapeutic effect for the above-described diseases, and may significantly improve the patient's medication compliance. In addition, the formulation may exhibit sufficient therapeutic effects against diseases mediated by acid pump antagonistic activity even at a low dose, so that side effects thereof may be reduced and the therapeutic effects thereof may be maximized.

The present disclosure provides a use of the formulation of the present disclosure for the prevention or treatment of diseases mediated by acid pump antagonistic activity.

The present disclosure provides a use of the formulation of the present disclosure in the manufacture of a medicament for the prevention or treatment of diseases mediated by acid pump antagonistic activity.

The present disclosure provides a method for preventing or treating diseases mediated by acid pump antagonistic activity, the method including administering an effective amount of the formulation of the present disclosure to a subject in need thereof.

The use of the formulation of the present disclosure and the preventive or therapeutic method using the formulation are as described above unless there are contradictions.

### Advantageous Effects

As described above, the pharmaceutical composition of the present disclosure can prolong the therapeutic effect for a long time by modifying the release of the active ingredient, thus improving the patient's medication compliance. Thus, the composition may be effectively used for a disease for which a drug needs to be taken for a long period of time or the blood concentration of the drug at a time when the patient cannot take the drug needs to be maintained at a certain level or higher.

In addition, since the pharmaceutical composition of the present disclosure may contain an organic acid, it may increase the solubility of tegoprazan in the intestinal tract, thereby maximizing the effect of treating diseases mediated by acid pump antagonistic activity, and exhibiting sufficient stability.

In addition, since the formulation of the present disclosure includes a modified-release pharmaceutical composition and an immediate-release pharmaceutical composition in a single dosage form, it may stably maintain the blood concentration of a therapeutically effective amount of the active ingredient for a long period of time, and thus may be effectively used for the treatment of diseases mediated by acid pump antagonistic activity.

In addition, since the formulation of the present disclosure includes a modified-release pharmaceutical composition and an immediate-release pharmaceutical composition in a single dosage form, the process time for production thereof can be shortened and the efficiency of the process can be increased.

### DESCRIPTION OF DRAWINGS

FIG. 1 illustrates a schematic view of a tablet according to the present disclosure.

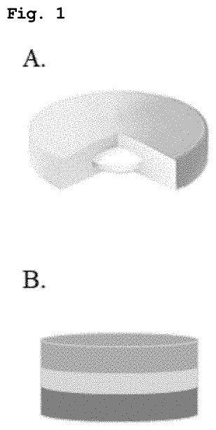

FIG. 2 shows changes in plasma concentrations of tegoprazan in beagles after oral administration of the modified-released formulation of the present disclosure and a commercially available K-CAB® tablet and an immediate-release formulation.

FIG. 3 shows changes in plasma concentrations of tegoprazan in mini-pigs after oral administration of a formulation containing a sugar-based sucrose as an inert particle and a formulation containing an organic acid according to the present disclosure.

FIG. 4 shows changes in blood concentrations of tegoprazan in monkeys after oral administration of a formulation containing a sugar-based sucrose as an inert particle and a formulation containing an organic acid according to the present disclosure.

### MODE FOR INVENTION

Hereinafter, the present disclosure will be described in more detail with reference to examples. However, these examples serve to illustrate the present disclosure, and the scope of the present disclosure is not limited by these examples.

### Preparation Example

To determine a suitable coating solution composition enabling tegoprazan coating, coating solutions were prepared using an active ingredient and various types of pharmaceutical additives (binding agent, surfactant, anti-adhesion agent, plasticizer, etc.) and solvents.

TABLE 1

| | Coating | Coating | Coating | Coating | Coating | Coating | Coating |
|---|---|---|---|---|---|---|---|
| | solution 1 | solution 2 | solution 3 | solution 4 | solution 5 | solution 6 | solution 7 |
| Pharmaceutical ingredients | Batch weight (g) | | | | | | |
| Tegoprazan | 4.81 | 4.81 | 4.81 | 4.81 | 4.81 | 4.81 | 4.81 |
| Hydroxypropyl cellulose | 0.96 | 0.96 | 0.96 | — | — | — | — |
| Povidone | — | — | — | 0.96 | 0.96 | 0.96 | — |
| Hypromellose | — | — | — | — | — | — | 0.96 |
| Polyethylene glycol | 0.23 | 0.23 | 0.23 | 0.23 | 0.23 | 0.23 | 0.23 |
| Talc | 0.17 | 0.17 | 0.17 | 0.17 | 0.17 | 0.17 | 0.17 |
| Polysorbate 80 | 0.48 | 0.48 | 0.48 | 0.48 | 0.48 | 0.48 | 0.48 |
| Purified water | q.s. | — | — | q.s. | — | — | — |
| Isopropyl alcohol | — | q.s. | — | — | q.s. | — | — |
| Anhydrous ethanol | — | — | q.s. | — | — | q.s. | — |
| Coating solution | Suspended | Partially | Dissolved | Suspended | Partially | Dissolved | Suspended |
| appearance | | dissolved | | | dissolved | | |
| | | and | | | and | | |
| | | suspended | | | suspended | | |

It was confirmed that the above-prepared coating solutions were suspended, or partially dissolved and suspended, or dissolved depending on the pharmaceutically usable solvent used. Each of the prepared coating solutions was applied thinly to a 150-mm Petri dish and dried, and then the solvent was evaporated. In this state, each of the coating layers was observed, and as a result, it was confirmed that, in the case of the coating solution in a suspended form, a layer was formed in a state in which the active ingredient particles were contained in the matrix structure of the binding, and in the case of the coating solution in a dissolved form, a translucent or transparent layer was formed.

### Examples 1 to 3

Before coating with a active ingredient layer containing tegoprazan, a separate inert coating (coating layer) was performed with each of the compositions (hypromellose, polyethylene glycol, and talc) shown in Table 2 below in order to increase the stability of the active ingredient layer, ensure the efficient formation of the coating layer and increase abrasion resistance.

TABLE 2

| | | Example 1 | Example 2 | Example 3 |
|---|---|---|---|---|
| Pharmaceutical ingredients | | Batch weight (g) | | |
| Suglet | | 2,000.0 | 2,000.0 | 2,000.0 |
| Hypromellose | | 40.0 | 40.0 | 40.0 |
| Polyethylene glycol | | 8.0 | 8.0 | — |
| Talc | | 32.0 | 32.0 | 40.0 |
| Solvent | Purified water | 920.0 as mixture | 920 | 920 |
| | Anhydrous | | — | — |
| | ethanol | | | |

Specifically, spherical pellets (product name: Suglet, Colorcon) based on sucrose were used as inert particles, and a fluidized-bed pellet coater (GPCG-1, Bottom spray, Glatt, Germany) was used for coating. The operating conditions of the fluidized bed pellet coater were an air supply temperature of 60±10° C., an exhaust air flap pressure of 0.6±0.2 bar, and a coating solution spray pressure of 1.5±0.6 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±10° C. The drying process could be operated within the allowable residual solvent range depending on the binding agent used, and could be performed by a vacuum drying method or an oven drying method.

### Examples 4 to 6

For coating of an active ingredient layer containing tegoprazan, a coating solution having the composition shown in Table 3 below was prepared using pharmaceutically acceptable additives and solvents. Then, the outer surface of a certain amount or the entire amount of the process product containing Examples 1 or 3 was coated with an active ingredient layer using a fluidized-bed pellet coater (GPCG-1, Bottom spray, Glatt, GERMANY). At this time, coating was performed so that a single particle included 25 to 200 mg.

TABLE 3

| | | Example 4 | Example 5 | Example 6 |
|---|---|---|---|---|
| Pharmaceutical ingredients | | Batch weight (g) | | |
| | | Example 1 | Example 2 | Example 3 |
| Tegoprazan | | 250 | 528.85 | 480.77 |
| Povidone | | 50 | 105.77 | 96.15 |
| Polyethylene glycol | | 12 | — | — |
| Talc | | 9 | 21.15 | 72.12 |
| Polysorbate 80 | | 25 | 21.15 | 19.23 |
| Solvent | Purified water | — | 1992 | 2527 |
| | Anhydrous | 3114 | 408 | 517 |
| | ethanol | | | |

The operating conditions of the fluidized bed pellet coater used for the coating were an air supply temperature of 65±10° C., an exhaust air flap pressure of 0.7±0.3 bar, and a coating solution spray pressure of 1.5±0.7 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±10° C. The drying process could be operated within the allowable residual solvent range depending on the pharmaceutical binding agent used, and could be performed by a vacuum drying method or an oven drying method.

### Examples 7 to 9

To increase the stability of the active ingredient layer containing tegoprazan, ensure the efficient formation of the coating layer and increase abrasion resistance, coating solutions having the composition shown in Table 4 below were prepared, and then inert coating (coating layer) was performed on the outer surface of a certain amount or the entire amount of the process product containing each of Examples 4 to 6 using a fluidized-bed pellet coater (GPCG-1, Bottom spray, Glatt, Germany).

TABLE 4

| | | |
|---|---|---|
| Example 7 | Example 8 | Example 9 |

| | |
|---|---|
| Pharmaceutical ingredients | Batch weight (g) |

| | | Example 4 | Example 5 | Example 6 |
|---|---|---|---|---|
| Hypromellose 3 cps | | 31.96 | 61.35 | — |
| Hypromellose 6 cps | | — | — | 53.79 |
| Polyethylene glycol | | 7.39 | | |
| Talc | | 10.16 | 25.54 | 21.5 |
| Solvent | Purified water | 569.0 as mixture | 988.0 | 1180 |
| | Anhydrous | | — | — |
| | ethanol | | | |

The operating conditions of the fluidized bed pellet coater used for the coating were an air supply temperature of 60±10° C., an exhaust air flap pressure of 0.6±0.2 bar, and a coating solution spray pressure of 1.5±0.6 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±10° C. The drying process could be operated within the allowable residual solvent range depending on the pharmaceutical binding agent used, and could be performed by a vacuum drying method or an oven drying method.

### Examples 10 to 21

In order for a certain amount or the entire amount of the process product containing Example 7 or 8 to have a delayed modified-release (modified-release) form, coating solutions having the compositions shown in Tables 5 and 6 below were prepared using pharmaceutically acceptable additives and solvents, and then delayed modified-release pellets were prepared using a fluidized-bed pellet coater (GPCG-1, Bottom spray, Glatt, Germany).

TABLE 5

| | | Example 10 | Example 11 | Example 12 | Example 13 | Example 14 | Example 15 |
|---|---|---|---|---|---|---|---|
| Pharmaceutical ingredients | | Batch weight (g) | | | | | |
| | | Example 7 | Example 7 | Example 8 | Example 8 | Example 8 | Example 8 |
| Triethyl citrate | | 11.25 | 11.29 | 11.7 | 11.7 | 10.24 | 10.41 |
| Talc | | 9.3 | 12.85 | 25.19 | 25.19 | 22.04 | 22.4 |
| Polysorbate 80 | | 2.15 | 2.87 | 0.48 | 0.48 | 0.42 | 0.39 |
| Methacrylic acid-ethyl acrylate copolymer | | 45.02 | 72.03 | 90.01 | — | — | 79.99 |
| Methacrylic acid-methylmethacrylate | | 36.02 | 18.01 | — | 90.01 | — | — |
| copolymer (1:1) | | | | | | | |
| Methacrylic acid-methylmethacrylate | | 9 | — | — | — | 78.76 | — |
| copolymer (1:2) | | | | | | | |
| Solvent | Purified water | 779 as | 518 as | 852 | 115 | 100 | 757 |
| | Anhydrous ethyl | mixture | mixture | — | 1032 | 903 | — |
| | Isopropyl alcohol | — | — | — | — | — | — |
| | Acetone | — | — | — | — | — | — |

TABLE 6

| | | Example 16 | Example 17 | Example 18 | Example 19 | Example 20 | Example 21 |
|---|---|---|---|---|---|---|---|
| Pharmaceutical ingredients | | Batch weight (g) | | | | | |
| | | Example 8 | Example 8 | Example 8 | Example 8 | Example 8 | Example 8 |
| Triethyl citrate | | 10.41 | 10.41 | 10.41 | 10.41 | 13.01 | 13.01 |
| Talc | | 22.4 | 22.4 | 22.4 | 22.4 | 28 | 28 |
| Polysorbate 80 | | 0.39 | 0.39 | 0.39 | 0.39 | 0.48 | 0.48 |
| Methacrylic acid-ethyl acrylate copolymer | | — | — | 79.99 | 15.98 | 62.5 | 33.3 |
| Methacrylic acid-methylmethacrylate | | 79.99 | — | — | 64 | — | — |
| copolymer (1:1) | | | | | | | |
| Methacrylic acid-methylmethacrylate | | — | 79.99 | — | — | 37.53 | 66.7 |
| copolymer (1:2) | | | | | | | |
| Solvent | Purified water | 102 | 102 | 102 | 102 | 127 | 127 |
| | Anhydrous ethyl | 917 | 917 | — | — | 1146 | 1146 |
| | Isopropyl alcohol | — | — | 611 | 611 | — | — |
| | Acetone | — | — | 306 | 306 | — | — |

When the solvent was purified water, the operating conditions of the fluidized bed pellet coater used for the coating were an air supply temperature of 60±10° C., an exhaust air flap pressure of 40±10 bar, and a coating solution spray pressure of 1.5±0.6 bar, and when the solvent was a mixture of purified water, alcohol and acetone, the operating conditions of the fluidized bed pellet coater used for the coating were an air supply temperature of 35±10° C., an exhaust air flap pressure of 0.6±0.2 bar, and a coating solution spray pressure of 1.5±0.6 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±1° C. The drying process could be operated within the allowable residual solvent range depending on the pharmaceutical binding agent used, and could be performed by a vacuum drying method or an oven drying method.

### Experimental Example 1. Dissolution Evaluation Under Acidic Conditions

For dissolution evaluation, in order to exclude the effect of the disintegration or disruption time of the hard capsule, the pellets not filled in the capsule were weighed, and dissolution evaluation of Examples 8 to 10 was performed according to the United States Pharmacopeia (USP) apparatus 1 (basket).

Dissolution conditions were set as follows: pH 1.2 (hydrochloric acid buffer); 37±0.5° C.; 900 ml medium; 100 rpm. The sample solution obtained after the initiation of dissolution was analyzed using an ultraviolet spectrometer of high-performance liquid chromatography (HPLC; manufactured by Agilent Technologies).

TABLE 7

| | 5 | 10 | 15 | 30 | 45 | 60 |
|---|---|---|---|---|---|---|
| | min | min | min | min | min | min |
| Example 7 | 101.8 | 101.7 | 101.7 | 100.9 | 99.79 | 99.24 |
| Example 8 | 86.8 | 94.2 | 94.5 | 94.3 | 93.4 | 92.4 |
| Example 9 | 98.1 | 98.4 | 98.5 | 98.6 | 98.6 | 98.4 |

As can be seen in Table 7 above, it could be confirmed that, in the case of Examples 7 to 9 to which the delayed modified-release layer was not applied, tegoprazan was dissolved under the acidic medium condition within a short time, suggesting that the pellet of the present disclosure could be used as a pellet for immediate release of tegoprazan.

### Experimental Example 2. Evaluation of Acid Resistance

For dissolution evaluation, in order to exclude the effect of the disintegration time of the hard capsule, the pellets not filled in the capsule were weighed, and dissolution evaluation of Examples 10 to 21 was performed according to the United States Pharmacopeia (USP) apparatus 1 (basket) to evaluate the acid resistances thereof.

Dissolution conditions were set as follows: pH 1.2 (hydrochloric acid buffer); 37±0.5° C.; 900 ml medium; 100 rpm. The sample solution obtained after the initiation of dissolution was analyzed using an ultraviolet spectrometer of high-performance liquid chromatography (HPLC; manufactured by Agilent Technologies).

TABLE 8

| | 5 | 10 | 15 | 30 | 45 | 60 | 90 | 120 |
|---|---|---|---|---|---|---|---|---|
| | min | min | min | min | min | min | min | min |
| Example 10 | 0 | 0 | 0 | 0 | 0 | 0 | 0 | 0 |
| Example 11 | 0 | 0 | 0 | 0 | 0 | 0 | 0 | 0 |
| Example 12 | 0 | 0 | 0 | 0 | 0 | 0 | 0 | 0 |
| Example 13 | 0 | 0 | 0 | 0 | 0 | 0 | 2.1 | 4.7 |
| Example 14 | 0 | 0 | 0 | 0 | 0 | 0 | 0 | 0 |
| Example 15 | 0 | 0 | 0 | 0 | 0 | 0 | 0 | 0 |
| Example 16 | 0 | 0 | 0 | 0 | 0 | 0 | 0 | 1.2 |
| Example 17 | 0 | 0 | 0 | 0 | 0 | 0 | 0 | 0 |
| Example 18 | 0 | 0 | 0 | 0 | 0 | 0 | 0 | 0.4 |
| Example 19 | 0 | 0 | 0 | 0 | 0 | 0 | 0.3 | 1.3 |
| Example 20 | 0 | 0 | 0 | 0 | 0 | 0 | 0 | 0 |
| Example 21 | 0 | 0 | 0 | 0 | 0 | 0 | 0 | 0 |

As can be seen in Table 8 above, it could be confirmed that, unlike Examples 7 to 9 which showed high dissolution rate under the acidic medium condition because the delayed modified-release layer was not included, in the case of Examples 10 to 21 including the delayed modified-release layer, tegoprazan was not dissolved within 2 hours (120 minutes) under the acidic medium condition, or only a low dissolution rate appeared after 90 minutes, suggesting that sufficient acid resistance was ensured.

### Experimental Example 3. Evaluation of Dissolution Under Weak Alkaline Condition

After completion of acid resistance evaluation in Experimental Example 2, dissolution evaluation for the pellets including the modified-release layer was performed in a weak alkaline medium.

The continuous dissolution test in this Experimental Example is a method by which a conventional technician or a person skilled in the art and related persons can evaluate a delayed modified-release formulation in laboratory or in-vitro. In this method, the dissolution in an acidic medium for a specified period of time is evaluated, and then the sample is transferred to a weak alkaline medium, or the pH in the medium is increased using an alkalizing agent. This method is used for the purpose of quality control and to evaluate in vitro behavior of drugs (Guidance for Industry, SUPAC-MR: Modified Release Solid Oral Dosage Form).

Specifically, after completion of acid resistance evaluation, the basket (apparatus 1) containing the pellets was set to the following dissolution conditions: preheated pH 6.8 (phosphate buffer); 37±0.5° C.; 900 ml medium; 100 rpm. The sample solution obtained after the initiation of dissolution was analyzed using an ultraviolet spectrometer of high-performance liquid chromatography (HPLC; manufactured by Agilent Technologies), and the results are shown in Table 9 below.

TABLE 9

| | 5 | 10 | 15 | 30 | 45 | 60 | 90 | 120 | 180 | 240 | 300 | 360 |
|---|---|---|---|---|---|---|---|---|---|---|---|---|
| | min | min | min | min | min | min | min | min | min | min | min | min |
| Example 10 | 0 | 0 | 3.3 | 11.4 | 18.0 | 23.8 | 33.0 | 41.0 | 51.7 | 60.9 | 69.0 | 75.5 |
| Example 12 | 1.9 | 27.6 | 45.5 | 64.0 | 72.8 | 76.8 | 79.9 | 82.7 | 85.2 | 86.5 | 86.8 | 89.1 |
| Example 13 | 0 | 3.7 | 22.9 | 55.9 | 70.3 | 76.1 | 81.0 | 83.0 | 85.0 | 86.8 | 86.5 | 87.6 |
| Example 15 | 1.3 | 27.4 | 48.9 | 64.6 | 74.3 | 78.2 | 80.5 | 81.8 | 82.8 | 83.7 | 83.7 | 83.7 |
| Example 19 | 2.3 | 7.4 | 21.4 | 47.7 | 59.3 | 65.4 | 72.6 | 75.8 | 79.0 | 80.2 | 81.6 | 82.1 |
| Example 20 | 0 | 0 | 6.1 | 33.3 | 54.8 | 67.0 | 81.0 | 83.5 | 85.7 | 86.7 | 87.5 | 88.5 |
| Example 21 | 0 | 0 | 0 | 1.9 | 30.0 | 42.1 | 61.0 | 73.3 | 82.2 | 84.9 | 86.5 | 87.7 |

### Examples 22 and 23

In order to increase the stability of the active ingredient layer, ensure the efficient formation of a coating layer and increase abrasion resistance, coating solutions having the compositions (hypromellose 3 cps, hypromellose 6 cps, talc, and solvent) shown in Table 10 below were prepared with additives and solvents, and then inert particles containing an organic acid were coated with each of the coating solution using a fluidized-bed pellet coater (GPCG-1, Bottom spray, Glatt, Germany) to form a separate isolation layer.

TABLE 10

| | |
|---|---|
| Example 22 | Example 23 |

| | |
|---|---|
| Pharmaceutical ingredients | Batch weight (g) |

| Tartaric add pellets | | 2000 | 2000 |
|---|---|---|---|
| Hypromellose 3 cps | | 40 | — |
| Hypromellose 6 cps | | — | 40 |
| Talc | | 40 | 40 |
| Solvent | Purified water | 920 | 46 |
| | Anhydrous | — | 874 |
| | ethanol | | |

The operating conditions of the fluidized bed pellet coater used for the coating were an air supply temperature of 60±10° C., an exhaust air flap pressure of 0.6±0.2 bar, and a coating solution spray pressure of 1.5±0.6 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±10° C. The drying process could be operated within the allowable residual solvent range depending on the pharmaceutical binding agent used, and could be performed by a vacuum drying method or an oven drying method.

### Examples 24 to 28

For coating with an active ingredient layer containing tegoprazan, coating solutions having the compositions shown in Table 11 below were prepared with pharmaceutical additives and solvents, and then the outer surface of a certain amount or the entire amount of the process product including Example 22 or 23 was coated with each of the coating solution using a fluidized bed pellet coater (GPCG-1, Bottom spray, Glatt, Germany).

TABLE 11

| | | Example 24 | Example 25 | Example 26 | Example 27 | Example 28 |
|---|---|---|---|---|---|---|
| Pharmaceutical ingredients | | Batch weight (g) | | | | |
| | | Example 22 | Example 23 | Example 23 | Example 23 | Example 23 |
| Tegoprazan | | 576.92 | 480.77 | 480.77 | 480.77 | 480.77 |
| Povidone | | 115.38 | 96.15 | 96.15 | 96.15 | 96.15 |
| Talc | | 23.08 | 19.23 | 48.08 | 72.12 | 96.15 |
| Polysorbate 80 | | 23.08 | 19.23 | 19.23 | 19.23 | 19.23 |
| Solvent | Purified water | 2173 | 2494 | 2436 | 2527 | 2628 |
| | Anhydrous ethanol | 445 | 511 | 499 | 518 | 536 |

The operating conditions of the fluidized bed pellet coater used for the coating were an air supply temperature of 60±10° C., an exhaust air flap pressure of 0.7±0.3 bar, and a coating solution spray pressure of 1.5±0.7 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±10° C. The drying process could be operated within the allowable residual solvent range depending on the pharmaceutical binding agent used, and could be performed by a vacuum drying method or an oven drying method.

### Examples 29 and 30

To increase the stability of the active ingredient layer containing tegoprazan, ensure the efficient formation of the coating layer and increase abrasion resistance, coating solutions having the composition shown in Table 12 below were prepared, and then inert coating (coating layer) was performed on the outer surface of a certain amount or the entire amount of the process product containing Example 24 to 25 using a fluidized-bed pellet coater (GPCG-1, Bottom spray, Glatt, Germany).

TABLE 12

| | |
|---|---|
| Example 29 | Example 30 |

| | |
|---|---|
| Pharmaceutical ingredients | Batch weight (g) |

| | | Example 24 | Example 25 |
|---|---|---|---|
| Hypromellose | | 66.92 | 66.92 |
| Talc | | 26.77 | 26.77 |
| Solvent | Purified water | 54 | 1467 |
| | Anhydrous | 1024 | — |
| | ethanol | | |

The operating conditions of the fluidized bed pellet coater used for the coating were an air supply temperature of 60±10° C., an exhaust air flap pressure of 0.6±0.2 bar, and a coating solution spray pressure of 1.5±0.6 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±10° C. The drying process could be operated within the allowable residual solvent range depending on the pharmaceutical binding agent used, and could be performed by a vacuum drying method or an oven drying method.

### Examples 31 to 38

In order for a certain amount or the entire amount of the process product containing Example 29 or 30 to have a delayed modified-release form, coating solutions having the compositions shown in Table 13 below were prepared with additives and solvents, and then delayed modified-release pellets were prepared using a fluidized-bed pellet coater (GPCG-1, Bottom spray, Glatt, Germany).

TABLE 13

| | | Example 31 | Example 32 | Example 33 | Example 34 | Example 35 | Example 36 | Example 37 | Example 38 |
|---|---|---|---|---|---|---|---|---|---|
| Pharmaceutical ingredients | | Batch weight (g) | | | | | | | |
| | | Example 29 | Example 29 | Example 29 | Example 29 | Example 29 | Example 29 | Example 30 | Example 30 |
| Triethyl citrate | | 7.8 | 10.41 | 10.41 | 10.41 | 10.41 | 10.41 | 13.0 | 13.0 |
| Talc | | 16.79 | 22.4 | 22.4 | 22.4 | 22.4 | 22.4 | 28 | 28 |
| Polysorbate 80 | | 0.32 | 0.39 | 0.39 | 0.39 | 0.39 | 0.39 | 0.5 | 0.5 |
| Methacrylic acid-ethyl | | 60.01 | 79.99 | — | — | 79.99 | 15.98 | 62.5 | 33.3 |
| acrylate copolymer | | | | | | | | | |
| Methacrylic acid- | | — | — | 79.99 | — | — | 64.0 | — | — |
| methylmethacrylate | | | | | | | | | |
| copolymer (1:1) | | | | | | | | | |
| Methacrylic acid- | | — | — | — | 79.99 | — | — | 37.53 | 66.7 |
| methylmethacrylate | | | | | | | | | |
| copolymer (1:2) | | | | | | | | | |
| Solvent | Purified water | 568 | 757 | 102 | 102 | 102 | 102 | 127 | 127 |
| | Anhydrous ethyl | — | — | 917 | 917 | — | — | 1146 | 1146 |
| | Isopropyl alcohol | — | — | — | — | 611 | 611 | — | — |
| | Acetone | — | — | — | — | 306 | 306 | — | — |

When the solvent was purified water, the operating conditions of the fluidized bed pellet coater used for the coating were an air supply temperature of 60±10° C., an exhaust air flap pressure of 40±10 bar, and a coating solution spray pressure of 1.5±0.6 bar, and when the solvent was a mixture of purified water, alcohol and acetone, the operating conditions of the fluidized bed pellet coater used for the coating were an air supply temperature of 35±10° C., an exhaust air flap pressure of 0.6±0.2 bar, and a coating solution spray pressure of 1.5±0.6 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±10° C. The drying process could be operated within the allowable residual solvent range depending on the pharmaceutical binding agent used, and could be performed by a vacuum drying method or an oven drying method.

### Experimental Example 4. Evaluation of Acid Resistance

For dissolution evaluation, in order to exclude the effect of the disintegration time of the hard capsule, the pellets not filled in the capsule were weighed, and dissolution evaluation of Examples 31, 32, 34, 37 and 38 was performed according to the United States Pharmacopeia (USP) apparatus 1 (basket) to evaluate the acid resistances thereof under an acidic condition.

Dissolution conditions were set as follows: pH 1.2 (hydrochloric acid buffer); 37±0.5° C.; 900 ml medium; 100 rpm. The sample solution obtained after the initiation of dissolution was analyzed using an ultraviolet spectrometer of high-performance liquid chromatography (HPLC; manufactured by Agilent Technologies).

TABLE 14

| | 5 | 10 | 15 | 30 | 45 | 60 | 90 | 120 |
|---|---|---|---|---|---|---|---|---|
| | min | min | min | min | min | min | min | min |
| Example 31 | 0 | 0 | 0 | 0 | 0 | 0 | 3.4 | 5.2 |
| Example 32 | 0 | 0 | 0 | 0 | 0 | 0.6 | 4.3 | 7.4 |
| Example 34 | 0 | 0 | 0 | 0 | 0 | 0 | 0.3 | 0.7 |
| Example 37 | 0 | 0 | 0 | 0 | 0 | 0 | 0 | 0 |
| Example 38 | 0 | 0 | 0 | 0 | 0 | 0 | 0 | 0 |

As can be seen in Table 14 above, it could be confirmed that, in the case of Examples 31, 32, 34, 37 and 38 including the delayed modified-release layer, tegoprazan was not dissolved within 2 hours (120 minutes) under the acidic medium condition, or only a low dissolution rate appeared after 90 minutes, suggesting that sufficient acid resistance was ensured.

### Experimental Example 5. Evaluation of Dissolution Under Weak Alkaline Condition

After completion of acid resistance evaluation in Experimental Example 4, dissolution evaluation for the delayed modified-release pellets was performed in a weak alkaline medium.

Specifically, after completion of acid resistance evaluation, the basket (apparatus 1) containing the pellets was set to the following dissolution conditions: preheated pH 6.8 (phosphate buffer); 37±0.5° C.; 900 ml medium; 100 rpm. The sample solution obtained after the initiation of dissolution was analyzed using an ultraviolet spectrometer of high-performance liquid chromatography (HPLC; manufactured by Agilent Technologies), and the results are shown in Table 15 below.

TABLE 15

| | 5 | 10 | 15 | 30 | 45 | 60 | 90 | 120 | 180 | 240 | 300 | 360 |
|---|---|---|---|---|---|---|---|---|---|---|---|---|
| | min | min | min | min | min | min | min | min | min | min | min | min |
| Example 32 | 1.6 | 23.8 | 41.7 | 60.3 | 73.3 | 80.3 | 85.6 | 86.9 | 88.4 | 89.1 | 90.6 | 92.4 |
| Example 34 | 0 | 0 | 1.3 | 3.3 | 5.6 | 8.1 | 13.3 | 18.3 | 26.8 | 33.9 | 40.0 | 44.0 |
| Example 37 | 0 | 0 | 0 | 31.9 | 47.1 | 52.5 | 58.4 | 61.6 | 66.2 | 68.6 | 70.3 | 71.5 |
| Example 38 | 0 | 0 | 0 | 0 | 13.5 | 42.3 | 53.2 | 58.0 | 62.8 | 64.7 | 65.9 | 66.7 |

### Experimental Example 6. Evaluation of Pharmacokinetic Absorption Affect in Beagles

In the present disclosure, all experimental procedures regarding the evaluation of pharmacokinetic characteristics in non-clinical models were performed following the regulations of the Animal Experimental Ethics Committee (IACUC, Institutional Animal Care and Use Committee), and performed in consideration of human equivalent doses (HEDs).

For evaluation of the in vivo pharmacokinetic characteristics of the pellets (test group) of Examples 12 to 14, commercially available K-CAB® tablet (control group 1) and Example 8 including no modified-release layer (control group 2) were set as control groups. Hard capsules were filled with the pellets of each of the test group and control group 2 so that they contained tegoprazan at the same dose as the K-CAB® tablet, and then a non-clinical test was conducted.

The non-clinical model animals used in the test were a total of 15 beagles (20±2 months old males with an average weight of 13±2 kg) divided into each group, consisting of three animals. Each test drug was administered orally to the animals by single-dose parallel design in a fasting condition after fasting for at least 12 hours the day before administration. Before administration (0 hours) and at 0.25, 0.5, 0.75, 1, 2, 3, 4, 6, 8, 12 and 24 hours after administration, blood was collected from the cephalic vein using a disposable 3 ml syringe. As a blood sample container, a 4 ml sodium heparin tube (BD Biosciences, USA) containing an anticoagulant was used. The collected blood was centrifuged at 3,000 rpm for 15 minutes to isolate plasma, and the plasma was stored in a cryogenic (−70° C.) freezer until analysis.

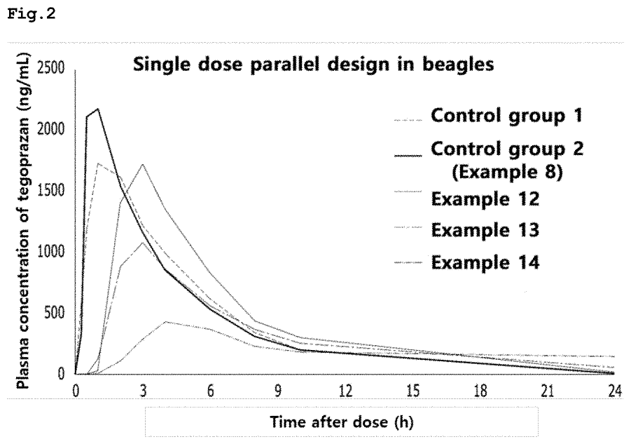

As an analysis instrument, a liquid chromatography mass spectrometer (LC-MS) was used, and analysis was performed in in-house method validation and electrospray ionization modes. The results are shown in Table 16 and FIG. 2.

TABLE 16

| PK | Control 1 | Control 2 | Example 12 | Example 13 | Example 14 |
|---|---|---|---|---|---|
| Tₘₐₓ(h)¹⁾ | 1.3 ± 0.6 | 0.7 ± 0.3 | 3.0 ± 0.0 | 2.7 ± 0.6 | 4.0 ± 0.0 |
| T_{1/2}(h)²⁾ | 3.5 ± 0.2 | 3.1 ± 0.5 | 3.5 ± 0.5 | 7.6 ± 5.7 | 4.0 ± 0.9 |
| Cₘₐₓ(ng/mL)³⁾ | 1840 ± 199 | 2400 ± 495 | 1720 ± 440 | 1130 ± 336 | 428 ± 141 |
| AUGₜ(ng · h/mL)⁴⁾ | 9850 ± 1200 | 9750 ± 3300 | 10300 ± 4290 | 7620 ± 2540 | 3630 ± 1910 |

¹⁾Tₘₐₓ: the time taken to reach Cₘₐₓ;
²⁾T_{1/2}: the time taken for drug concentration to decrease to half the original concentration;
³⁾Cₘₐₓ: the maximum concentration of the drug after drug administration;
⁴⁾AUCₜ: Area under the plasma concentration-time curve from administration time to the last sampling time t

As shown in Table 16 above and FIG. 2, it could be confirmed that control group 2 showed a faster Tₘₐₓ and a higher Cₘₐₓ value than control group 1 due to an increase in surface area after disintegration or dissolvation of the hard capsule film in the body, suggesting that it showed high in vivo exposure within a short time after administration. In addition, it could be confirmed that Examples 12 to 14 showed different in vivo absorption rates due to the pH-dependent physical properties of the enteric agent used for modified-release, and Examples 12 to 14 including the delayed modified-release layer showed a delay in the Tₘₐₓ value of the plasma concentration of tegoprazan compared to the control groups.

### Experimental Example 7. Evaluation of Pharmacokinetic Absorption Affect in Mini-Pigs According to Type of Inert Particles

For evaluation of in vivo pharmacokinetic absorption affect according to the type of inert particles, hard capsules were filled with the pellets of each of Examples 15, 17, 32 and 34 so that they could contain same dose of tegoprazan, and then a non-clinical test was conducted.

Mini-pigs having a relatively long gastrointestinal (GI) tract compared to beagles were selected as a non-clinical model, and comparative evaluation was performed using single-dose parallel design method.

mini-pigs (8 to 11 months old males with an average weight of 25.9±1.4 kg) divided into each group, consisting of three animals, were used in the test. Each test drug was administered orally to the animals by single-dose parallel design method in a fasting condition after fasting for at least 12 hours the day before administration.

At 0.5, 1, 1.5, 2, 3, 4, 5, 6, 7, 8, 12 and 24 hours after administration, about 3 ml of blood was collected from the jugular vein using a disposable syringe. As a blood sample container, a heparinized tube (5 IU/mL) was used. The collected blood was centrifuged at 3,000 rpm for 5 minutes to isolate plasma, and the plasma was stored in a cryogenic (−70° C.) freezer until analysis.

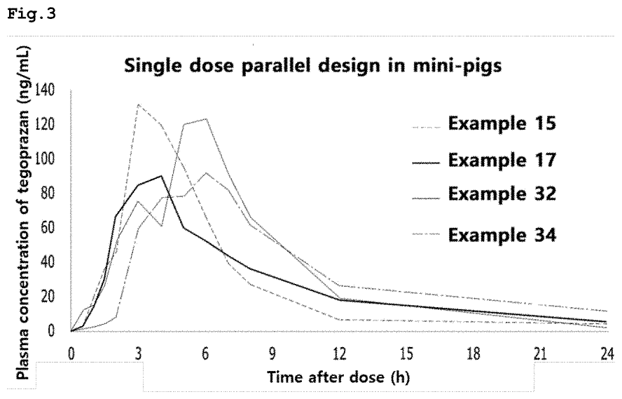

The plasma concentration of the drug was analyzed using LC-MS/MS in in-house method validation and electrospray ionization modes, and the results are shown in Table 17 below and FIG. 3.

TABLE 17

| Inert pellet | Sugar-based sucrose inert particle | | Organic add-containing inert particle | |
|---|---|---|---|---|
| PK | Example 15 | Example 17 | Example 32 | Example 34 |
| Tₘₐₓ | 3.7 ± 1.2 | 4.3 ± 1.5 | 4.7 ± 1.5 | 4.7 ± 1.5 |
| Cₘₐₓ(ng/mL) | 158 ± 75.6 | 108 ± 105 | 160 ± 43.5 | 115 ± 34.4 |
| AUGₜ(ng · h/mL) | 632 ± 137 | 671 ± 546 | 843 ± 240 | 836 ± 103 |

As shown in Table 17 and FIG. 3, the in vivo exposure of the modified-release formulation containing tegoprazan was higher in Examples 32 and 34, in which the inert particles containing organic acids were used, among the two types of inert particles used in the experiment.

There is a slight difference in intestinal length and intestinal pH between animal species, but mini-pigs are close to human-like environments. Thus, the above results suggest that, when inert particles containing an organic acid are used in the pharmaceutical composition of the present disclosure, the in vivo absorption rate of tegoprazan can be increased.

### Experimental Example 8. Evaluation of Pharmacokinetic Absorption Affect in Monkeys According to Type of Inert Particles

Monkeys which are the animal model most similar to humans in terms of anatomy, physiology and endocrinology were used as an animal model. Using this animal model, pharmacokinetic absorption affect according to the types of inert particles were comparatively evaluated by single dose parallel design method.

For evaluation of in vivo pharmacokinetic absorption affect, hard capsules were filled with the pellets of each of Examples 15 and 32 so that they could contain uniform amounts of tegoprazan, and then a non-clinical test was conducted.

The non-clinical model animals used in the test were a total of 6 cynomolgus monkeys (30 to 50 months old males with an average weight of 3.19±0.37 kg) divided into each group, consisting of three animals. Each test drug was administered orally to the animals by single-dose parallel design method in a fasting condition after fasting for at least 16 hours the day before administration.

At 0.5, 1, 1.5, 2, 3, 4, 5, 6, 8, 10, 12 and 24 hours after oral administration, about 1 ml of blood was collected from the femoral vein using a disposable syringe. As a blood sample container, BD Microtainer® (Sodium Heparin tube, BD Biosciences, USA) was used. The collected blood was centrifuged at 3,000 rpm at 4° C. for 15 minutes to isolate plasma, and the plasma was stored in a cryogenic (−70° C.) freezer until analysis.

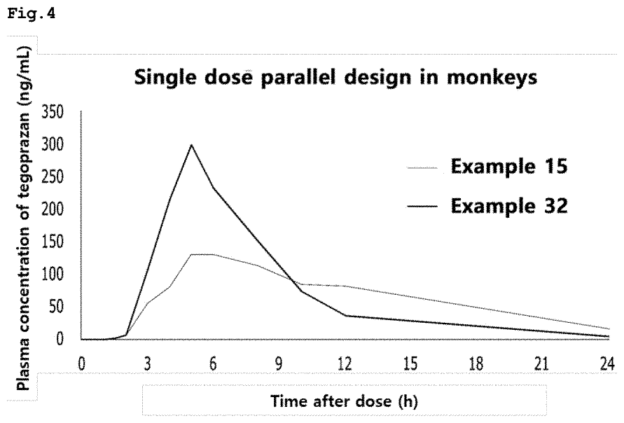

The plasma concentration of the drug was analyzed using LC-MS/MS in in-house method validation and electrospray ionization modes, and the results are shown in Table 18 below and FIG. 4.

TABLE 18

| PK | Example 15 | Example 32 |
|---|---|---|
| Tₘₐₓ(h) | 4.5 ± 0.7 | 4.3 ± 0.6 |
| T_{1/2}(h) | 6.3 ± 3.1 | 5.8 ± 4.8 |
| Cₘₐₓ(ng/mL) | 145 ± 25 | 362 ± 143 |
| AUGₜ(ng · h/mL) | 1520 ± 280 | 1700 ± 990 |

As shown in Table 18 and FIG. 4, the in vivo exposure of the modified-release formulation containing tegoprazan was higher in Examples 32, in which the inert particles containing organic acids were used, among the two types of inert particles used in the experiment.

The above results suggest that, when inert particles containing an organic acid are used, the in vivo absorption rate of tegoprazan can be increased.

Experimental Example 9. Evaluation of Pharmacokinetic Absorption Affect in Monkeys by Single Dose Parallel Design

A series of studies and analysis related experimental items for evaluating the pharmacokinetic absorption affect of immediate-release and modified-release pellets in monkeys by single dose parallel design method were conducted under conditions similar to those of Experimental Example 7. Monkeys (average weight: 4.48±0.56 kg; 4±2 years old) were divided into each group, consisting of 6 animals, and each drug was administered to each animal group. The results are shown in Table 19 below.

TABLE 19

| PK | Example 8 | Example 20 | Example 21 |
|---|---|---|---|
| Tₘₐₓ(h) | 1.7 ± 0.9 | 3.2 ± 0.8 | 3.4 ± 1.4 |
| Cₘₐₓ(ng/mL) | 274 ± 108 | 170 ± 35.8 | 123 ± 22.1 |
| AUGₜ(ng · h/mL) | 1260 ± 251 | 1420 ± 286 | 1200 ± 286 |

As shown in Table 19, it was confirmed that Tₘₐₓ was delayed in Examples 20 and 21 including the delayed modified-release layer, compared to Example 8 not including the delayed modified-release layer, suggesting that the formulation including the delayed modified-release layer showed a modified-release pattern. In addition, it was confirmed that Example 20 and 21 which are delayed modified-release formulations were similar to Example 8 which is an immediate-release formulation in the result of in vivo exposure of the drug, and among the pharmacokinetics (PK) of tegoprazan according to the release pattern of the formulation, the area under the concentration-time curve (AUC) was similar between the Examples.

### Examples 39 to 46

For coating with the active ingredient layer containing tegoprazan, coating solutions having the pharmaceutical additive compositions shown in Table 20 below were prepared (solvent: q.s.) with various binding agents, and then sucrose-based inert pellets (product name: Suglet, Colorcon) were coated with each of the coating solution using a fluidized bed pellet coater (GPCG-1, Bottom spray, Glatt, Germany).

TABLE 20

| | | Example | Example | Example | Example | Example | Example | Example | Example |
|---|---|---|---|---|---|---|---|---|---|
| | | 39 | 40 | 41 | 42 | 43 | 44 | 45 | 46 |
| Suglet 25/30 | | 52.00 | 100.00 | 100.00 | 100.00 | 100.00 | 100.00 | 100.00 | 100.00 |
| Tegoprazan | Tegoprazan | 50.00 | 50.00 | 50.00 | 50.00 | 50.00 | 50.00 | 50.00 | 50.00 |
| layer | Povidone K90 | 10.00 | 10.00 | — | — | — | — | — | — |
| | Polyvinyl alcohol | — | — | 10.00 | — | 5.00 | — | — | — |
| | (EG-40) | | | | | | | | |
| | Hydroxypropyl | — | — | — | 10.00 | — | — | — | — |
| | cellulose | | | | | | | | |
| | (EXF) | | | | | | | | |
| | Polyethylene | — | — | — | — | — | 10.00 | — | — |
| | glycol•polyvinyl | | | | | | | | |
| | alcohol copolymer | | | | | | | | |
| | Hydroxypropyl | — | — | — | — | — | — | 10.00 | — |
| | methylcellulose | | | | | | | | |
| | (15 cps) | | | | | | | | |
| | Hydroxypropyl | — | — | — | — | — | — | — | 10.00 |
| | cellulose | | | | | | | | |
| | (GXF) | | | | | | | | |
| | Polysorbate 80 | 2.00 | — | — | — | — | — | — | — |
| | Talc | 7.50 | 7.50 | 7.50 | 7.50 | 7.50 | 7.50 | 7.50 | 7.50 |

| | | | | | | | | |
|---|---|---|---|---|---|---|---|---|
| Total | 121.50 | 167.50 | 167.50 | 167.50 | 162.50 | 167.50 | 167.50 | 167.50 |

(unit: mg/capsule)

The content in Table 20 is expressed in terms of the amount (mg) of pellets filled per capsule.

The operating conditions of the fluidized bed pellet coater were an air supply temperature of 75±10° C., an exhaust air flap pressure of 0.7±0.3 bar, and a coating solution spray pressure of 1.5±0.7 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 75±10° C. The drying process could be operated within the allowable residual solvent range depending on the binding agent used, and could be performed by a vacuum drying method or an oven drying method.

### Experimental Example 10. Dissolution Evaluation

For dissolution evaluation, in order to exclude the effect of the disintegration time of the hard capsule, the pellets not filled in the capsule were weighed, and dissolution evaluation of Examples 39 to 44 was performed according to the United States Pharmacopeia (USP) apparatus 1 (basket).

Dissolution conditions were set as follows: pH 4.0 (acetate buffer); 37±0.5° C.; 900 mL medium; 50 rpm. The sample solution obtained after the initiation of dissolution was analyzed using an ultraviolet spectrometer of high-performance liquid chromatography (HPLC; manufactured by Agilent Technologies). The results are shown in Table 21 below.

TABLE 21

| | 5 | 10 | 15 | 30 | 45 | 60 | 90 | 120 |
|---|---|---|---|---|---|---|---|---|
| | min | min | min | min | min | min | min | min |
| Example 39 | 28.6 | 66.6 | 80.4 | 93.8 | 96.4 | 97.4 | 98.2 | 98.6 |
| Example 40 | 12.2 | 28.6 | 39.0 | 58.1 | 69.1 | 76.1 | 83.6 | 87.9 |
| Example 41 | 16.9 | 55.7 | 73.0 | 83.9 | 86.4 | 87.8 | 89.5 | 90.5 |
| Example 42 | 40.3 | 64.7 | 71.7 | 79.4 | 82.9 | 85.0 | 87.4 | 88.7 |
| Example 43 | 2.3 | 10.0 | 20.9 | 43.3 | 58.7 | 69.0 | 83.2 | 90.6 |
| Example 44 | 63.6 | 81.2 | 89.2 | 98.0 | 99.5 | 99.5 | 99.7 | 99.5 |

As shown in Table 21 above, it can be seen that the release rate of tegoprazan can be modified depending on the type of a polymer. Specifically, it can be seen that the release rate of the active ingredient decreased as the viscosity of the polymer used increased, and that Example 43 (using 5.0 mg PVA) had the lowest release rate, and Example 44 (using 10.0 mg polyethylene glycol) had the highest release rate.

### Examples 47 to 55

For coating with an active ingredient layer containing tegoprazan, coating solutions having the compositions shown in Table 22 below were prepared with pharmaceutical additives and solvents, and then inert pellets were coated with each of the coating solutions. Coating was performed under substantially the same conditions and using the same method as in Examples 39 to 47.

TABLE 22

| | | Example | Example | Example | Example | Example | Example | Example | Example | Example |
|---|---|---|---|---|---|---|---|---|---|---|
| | | 47 | 48 | 49 | 50 | 51 | 52 | 53 | 54 | 55 |
| | | mg/capsule | | | | | | | | |
| Suglet 25/30 | | 50.00 | 50.00 | 50.00 | 50.00 | 50.00 | 50.00 | 50.00 | 50.00 | 50.00 |
| Tegoprazan | Tegoprazan | 50.00 | 50.00 | 50.00 | 50.00 | 50.00 | 50.00 | 50.00 | 50.00 | 50.00 |
| layer | Polyvinyl | 5.00 | 2.50 | 5.00 | 5.00 | 5.00 | 2.50 | 2.50 | 3.75 | 2.50 |
| | alcohol | | | | | | | | | |
| | (EG-40) | | | | | | | | | |
| | Talc | 22.50 | 7.50 | 7.50 | 7.50 | 22.50 | 22.50 | 7.50 | 15.00 | 22.50 |
| | Purified | 310.00 | 240.00 | 250.00 | 145.83 | 180.83 | 175.00 | 140.00 | 206.25 | 300.00 |
| | water | | | | | | | | | |

| | | | | | | | | | |
|---|---|---|---|---|---|---|---|---|---|
| Total | 127.50 | 110.00 | 112.50 | 112.50 | 127.50 | 125.00 | 110.00 | 118.75 | 125.00 |

### Experimental Example 11. Dissolution Evaluation

Dissolution evaluation of Examples 47 to 55 was performed under substantially the same conditions and using the same method as in Experimental Example 9. The results are shown in Table 23.

TABLE 23

| | 5 | 10 | 15 | 30 | 45 | 60 | 90 | 120 |
|---|---|---|---|---|---|---|---|---|
| | min | min | min | min | min | min | min | in |
| Example 47 | 0.4 | 2.5 | 3.9 | 8.7 | 15.0 | 21.5 | 31.9 | 40.1 |
| Example 48 | 1.7 | 4.1 | 6.6 | 13.1 | 19.3 | 24.6 | 33.2 | 40.9 |
| Example 49 | 1.2 | 4.7 | 9.6 | 29.3 | 45.3 | 57.5 | 74.5 | 84.3 |
| Example 50 | 2.6 | 10.0 | 22.8 | 49.5 | 65.6 | 74.0 | 82.3 | 87.0 |
| Example 51 | 1.3 | 3.0 | 4.6 | 13.6 | 22.9 | 31.2 | 44.1 | 54.5 |
| Example 52 | 1.7 | 3.3 | 4.9 | 9.7 | 15.5 | 20.6 | 29.1 | 36.3 |
| Example 53 | 3.8 | 9.3 | 15.0 | 29.6 | 42.1 | 52.3 | 67.4 | 79.0 |
| Example 54 | 1.3 | 2.9 | 4.3 | 9.0 | 14.4 | 20.3 | 31.8 | 42.7 |
| Example 55 | 1.4 | 2.7 | 3.9 | 7.2 | 10.1 | 12.9 | 17.2 | 20.7 |

As shown in Table 23 above, it can be seen that the release rate of tegoprazan can be modified depending on the amount of talc. In particular, it can be seen that the batch of Example 55 (using an excessive amount (22.5 mg) of talc) had the lowest release rate, and the batch of Example 50 (using a small amount (7.5 mg) of talc) had the highest release rate.

### Examples 56 and 61

To increase the stability of the active ingredient layer containing tegoprazan and the pellets including the same, ensure the efficient formation of the coating layer and increase abrasion resistance, coating solutions having the compositions shown in Table 24 below were prepared, and then the outer surface of a certain amount or the entire amount of the process product containing Example 39, 50 or 51 was coated with each of the coating solutions using a fluidized-bed pellet coater (GPCG-1, Bottom spray, Glatt, Germany).

TABLE 24

| | Example | Example | Example | Example | Example | Example |
|---|---|---|---|---|---|---|
| | 56 | 57 | 58 | 59 | 60 | 61 |
| | mg/capsule | | | | | |
| Active ingredient | Example 39 | Example 39 | Example 39 | Example 39 | Example 51 | Example 50 |
| coating pellet | 121.50 | 121.50 | 121.50 | 121.50 | 127.50 | 112.50 |
| Hydroxypropyl methyl | — | — | — | — | 2.55 | 2.25 |
| cellulose (3 cps) | | | | | | |
| Ethyl cellulose | 16.63 | 12.15 | 18.23 | 24.30 | — | — |
| Polyethylene | 2.91 | 2.13 | 3.20 | 4.26 | — | — |
| glycol•polyvinyl | | | | | | |
| alcohol copolymer | | | | | | |
| Triethyl citrate | 4.15 | 3.04 | 4.56 | 6.08 | — | — |
| Talc | 4.74 | 1.73 | 2.60 | 3.46 | 2.55 | 2.25 |
| Purified water | 55.42 | 40.50 | 60.75 | 81.00 | 58.65 | 51.75 |
| Total | 149.93 | 140.55 | 150.09 | 159.60 | 132.60 | 117.00 |

The operating conditions of the fluidized bed pellet coater were an air supply temperature of 50±10° C., an exhaust air flap pressure of 0.6±0.2 bar, and a coating solution spray pressure of 1.5±0.6 bar. During the coating solution spraying process, the height of the partition and the feed rate of the coating solution were appropriately adjusted while observing fluidization according to the amount of pellets charged.

After completion of the coating solution spraying, curing was performed in the fluidized bed pellet coater for about 30 to 120 minutes while the pellets were fluidized by supplying air at 75±10° C. The drying process could also be performed by an oven drying method.

### Experimental Example 12. Dissolution Evaluation

For dissolution evaluation, in order to exclude the effect of the disintegration time of the hard capsule, the pellets not filled in the capsule were weighed, and dissolution evaluation of Examples 39 and 56 to 59 was performed according to the United States Pharmacopeia (USP) apparatus 1 (basket).

Dissolution conditions were set as follows: pH 6.8 (phosphate buffer); 37±0.5° C.; 900 mL medium; and 100 rpm. The sample solution obtained after the initiation of dissolution was analyzed using an ultraviolet spectrometer of high-performance liquid chromatography (HPLC; manufactured by Agilent Technologies), and the results are shown in Table 25 below.

TABLE 25

| | 15 | 30 | 45 | 60 | 90 | 120 | 240 | 360 | 480 | 720 |
|---|---|---|---|---|---|---|---|---|---|---|
| | min | min | min | min | min | min | min | min | min | min |
| Example 39 | 62.4 | 81.8 | 90.6 | 95.0 | 98.3 | 99.1 | 99.3 | 99.5 | 99.0 | 98.9 |
| Example 56 | 17.2 | 45.0 | 60.1 | 68.5 | 77.7 | 82.9 | 93.0 | 98.0 | 100.7 | 102.8 |
| Example | 26.8 | 52.3 | 67.3 | 76.8 | 86.4 | 91.5 | 98.8 | 102.5 | 104.0 | 105.6 |
| 57(10%) | | | | | | | | | | |
| Example | 14.2 | 34.5 | 46.4 | 54.5 | 64.4 | 70.3 | 81.1 | 87.1 | 90.0 | 94.1 |
| 58(15%) | | | | | | | | | | |
| Example | 9.2 | 28.2 | 39.8 | 48.1 | 58.8 | 65.2 | 76.0 | 81.6 | 84.4 | 88.2 |
| 59(20%) | | | | | | | | | | |

As shown in Table 25 above, it can be seen that the release rate can be modified according to the presence or absence of the coating layer and the composition of the coating layer. Specifically, it can be seen that, as the amount of the coating layer increases, the release rate of the active ingredient decreases.

Therefore, it can be confirmed that the pharmaceutical composition of the present disclosure can release tegoprazan in a sustained manner by modifying the release rate of tegoprazan, and in particular, it can be confirmed that the pharmaceutical composition may release tegoprazan in a sustained manner even in the intestinal environment.

Therefore, it can be seen that the pharmaceutical composition of the present disclosure may have a modified-release pattern in which tegoprazan is released in a sustained manner in a region ranging from the gastric juice environment to the intestinal environment.

### Examples 62 to 69

Enteric coating solutions having the compositions shown in Table 26 below were prepared with pharmaceutically acceptable additives and solvents, and then a certain amount or the entire amount of the process product including Example 56, 67 or 61 was coated with each of the enteric coating solutions using a fluidized bed pellet coater (GPCG-1, Bottom spray, Glatt, Germany).

TABLE 26

| | Example | Example | Example | Example | Example | Example | Example | Example |
|---|---|---|---|---|---|---|---|---|
| | 62 | 63 | 64 | 65 | 66 | 67 | 68 | 69 |
| | mg/capsule | | | | | | | |
| Active ingredient | Example 56 | Example 60 | Example 61 | Example 61 | Example 61 | Example 60 | Example 60 | Example 60 |
| coating pellet | 149.93 | 132.60 | 117.00 | 117.00 | 117.00 | 132.60 | 132.60 | 132.60 |
| Methacrylic acid | 10.00 | 13.26 | 17.55 | 23.40 | 35.10 | 19.89 | 26.52 | 39.78 |
| copolymer S | | | | | | | | |
| Methacrylic acid | 5.00 | 6.63 | — | — | — | — | — | — |
| copolymer L | | | | | | | | |
| Potassium hydroxide | — | — | 0.39 | 0.52 | 0.79 | 0.45 | 0.59 | 0.89 |
| (KOH) | | | | | | | | |
| Triethyl citrate | 3.00 | 1.99 | 10.53 | 14.04 | 21.06 | 11.93 | 15.91 | 23.87 |
| Talc | 7.50 | 9.95 | 8.78 | 11.70 | 17.55 | 9.95 | 13.26 | 19.89 |
| Purified water | 12.62 | 11.67 | 148.99 | 198.66 | 297.98 | 168.86 | 225.14 | 337.72 |
| Anhydrous ethanol | 216.79 | 221.71 | — | — | — | — | — | — |
| Total | 175.43 | 164.43 | 154.25 | 166.66 | 191.50 | 174.82 | 188.88 | 217.03 |

The operating conditions of the fluidized bed pellet coater used for the coating were an air supply temperature of 35±10° C., an exhaust air flap pressure of 0.7±0.3 bar, and a coating solution spray pressure of 1.5±0.7 bar. During the coating solution spraying process, the height of the partition and the feed rate of the coating solution were appropriately adjusted while observing fluidization according to the amount of pellets charged.

After completion of the coating solution spraying, drying was performed in the fluidized bed pellet coater for about 30 to 120 minutes while the pellets were fluidized by supplying air at 40±10° C. The drying process could also be performed by a vacuum drying method or an oven drying method.

### Experimental Example 13. Dissolution Evaluation

Dissolution evaluation of Examples 62 to 69 was performed under substantially the same conditions and using the same method as in Experimental Example 12. The results are shown in Table 27.

TABLE 27

| | 15 | 30 | 45 | 60 | 90 | 120 | 240 | 360 | 480 | 720 |
|---|---|---|---|---|---|---|---|---|---|---|
| | min | min | min | min | min | min | min | min | min | mim |
| Example 62 | 0.0 | 3.5 | 10.2 | 16.0 | 25.2 | 32.0 | 50.0 | 57.7 | 61.7 | 66.6 |
| Example 63 | 0.0 | 0.0 | 0.0 | 1.0 | 7.2 | 20.2 | 39.9 | 45.4 | 49.1 | 54.3 |
| Example | 0.0 | 2.2 | 4.1 | 6.4 | 10.4 | 17.2 | 41.2 | 53.6 | 59.1 | 64.4 |
| 64(15%) | | | | | | | | | | |
| Example | 0.0 | 1.7 | 2.8 | 4.6 | 9.0 | 13.2 | 40.1 | 52.6 | 58.2 | 64.8 |
| 65(20%) | | | | | | | | | | |
| Example | 0.0 | 0.0 | 0.9 | 2.1 | 3.5 | 5.1 | 12.4 | 27.7 | 37.8 | 53.1 |
| 66(30%) | | | | | | | | | | |
| Example | 0.4 | 3.3 | 6.8 | 10.7 | 17.0 | 21.8 | 37.5 | 44.0 | 48.0 | 53.0 |
| 67(15%) | | | | | | | | | | |
| Example | 0.0 | 0.8 | 2.9 | 4.8 | 8.9 | 13.4 | 36.4 | 47.9 | 52.7 | 56.6 |
| 68(20%) | | | | | | | | | | |
| Example | 0.0 | 0.0 | 0.0 | 0.4 | 1.3 | 3.2 | 11.6 | 22.2 | 31.3 | 49.1 |
| 69(30%) | | | | | | | | | | |

As shown in Table 27, it can be seen that, in the case of Examples 62 to 69, to which sustained-release and delayed modified-release were applied, tegoprazan was released for 720 minutes or more in a weak alkaline medium condition. That is, it can be seen that the pellets of the present disclosure can be used as delayed and modified-release pellets that release tegoprazan in the intestinal environment and release tegoprazan in a sustained manner.

In addition, as shown in Table 27, it can be seen that both the batch using the organic solvent and the batch using only purified water achieve similar sustained-release properties, and both the solution compositions can be used.

Furthermore, when comparing Examples 64 to 66 and Examples 67 to 69, it can be seen that, as the enteric coating rate in the pellet increases, the total dissolution rate tends to decrease, and even in the case the pellets having similar enteric coating rates, if there is a difference in the release rate of the active ingredient, the total dissolution rate is different. Accordingly, it can be seen that the final dissolution rate can be adjusted as desired by modifying the release rate of the active ingredient and the composition of the enteric coating layer.

Therefore, it can be seen that the pharmaceutical composition of the present disclosure can be used as a modified-release formulation that modifies the release of tegoprazan so as to have a desired release (dissolution) rate.

### Examples 70 to 74

To provide a sustained and modified-release form, granules having the compositions shown in Table 28 below were prepared with pharmaceutical additives, and then tableted to produce sustained-release tablets.

TABLE 28

| | | Example | Example | Example | Example | Example |
|---|---|---|---|---|---|---|
| | Sustained-release | 70 | 71 | 72 | 73 | 74 |
| Process | formulation | mg/Tablet | | | | |
| Inside of | Tegoprazan | 25.00 | 25.00 | 25.00 | 25.00 | 25.00 |
| granule | Mannitol 160C | 25.00 | 25.00 | 25.00 | 25.00 | 25.00 |
| | Microcrystalline cellulose | 21.00 | 21.00 | 21.00 | 21.00 | 21.00 |
| | Croscarmellose sodium | 3.00 | 3.00 | 3.00 | 3.00 | 3.00 |
| Binding | Hydroxypropyl cellulose | 3.00 | 3.00 | 3.00 | 3.00 | 3.00 |
| solution | Purified water | 33.00 | 33.00 | 33.00 | 33.00 | 33.00 |
| Outside of | Polyethylene oxide | 22.00 | — | — | — | — |
| granule | Hydroxypropyl methyl | — | 22.00 | — | — | — |
| | cellulose (15,000 cps) | | | | | |
| | Hydroxypropyl methyl | — | — | 22.00 | 44.00 | 66.00 |
| | cellulose (100,000 cps) | | | | | |
| lubricant | Magnesium stearate | 1.00 | 1.00 | 1.00 | 1.00 | 1.00 |

| | | | | | |
|---|---|---|---|---|---|
| Total | 100.00 | 100.00 | 100.00 | 122.00 | 144.00 |

Specifically, according to the amounts shown in Table 28 above, tegoprazan was mixed with mannitol, microcrystalline cellulose and croscarmellose sodium, and then sieving was performed. After the sieved material was added to a high shear mixer (DIOSNA), the granulation process was performed while the prepared binding solution was added.

The prepared granules were dried, and then subjected to a milling process using a screen having an appropriate size. Extragranular excipients shown in Table 28 above were added to and mixed with the milled granules in the amounts shown in Table 28. After completion of the mixing process, a lubrication process was performed by adding sieved magnesium stearate to the mixture. The lubricated materials were tableted using an appropriate punch, thus preparing sustained-release tablets.

### Experimental Example 14. Dissolution Evaluation of Sustained-Release Tablets

Dissolution evaluation was performed the United States Pharmacopeia (USP) apparatus 2 (paddle) under the following conditions: pH 6.8 (phosphate buffer); 37±0.5° C.; 900 mL medium; and 50 rpm. The sample solution obtained after the initiation of dissolution was analyzed using an ultraviolet spectrometer of high-performance liquid chromatography (HPLC; Agilent Technologies), and the results are shown in Table 29 below.

TABLE 29

| | 15 | 30 | 45 | 60 | 90 | 120 | 240 | 360 | 480 | 720 |
|---|---|---|---|---|---|---|---|---|---|---|
| | min | min | min | min | min | min | min | min | min | min |
| K-CAB ® | 31.1 | 46.4 | 53.9 | 59.5 | 66.5 | 71.1 | 79.8 | 83.2 | 85.1 | 88.0 |
| 25 mg | | | | | | | | | | |
| Example 72 | 4.6 | 6.6 | 7.6 | 8.8 | 11.1 | 13.1 | 21.5 | 30.6 | 40.2 | 58.6 |
| Example 73 | 5.4 | 8.0 | 9.6 | 11.2 | 14.0 | 16.6 | 26.6 | 37.1 | 48.2 | 69.0 |
| Example 74 | 3.0 | 3.3 | 3.4 | 3.9 | 4.5 | 4.9 | 7.9 | 11.6 | 16.8 | 29.0 |

As shown in Table 29, it can be confirmed that the sustained-release tablets of Examples 72 to 74 released tegoprazan slowly and sustainedly in a weak alkaline environment. In addition, when comparing Examples 72 to 74, it can be seen that the release rate of tegoprazan could be modified according to the proportion of the sustained-release agent.

### Examples 75 and 76

Granules having the compositions shown in Table 30 below were prepared with pharmaceutical additives, and then tableted to prepare immediate-release tablets. Granules and tablets were prepared under substantially the same conditions and using the same method as in Examples 70 to 74, except that the components and contents shown in Table 30 below were used.

TABLE 30

| | | Example 75 | Example 76 |
|---|---|---|---|
| Process | Components | mg/Dosage unit | |
| Inside of | Tegoprazan | 50.00 | 50.00 |
| granule | Mannitol | 50.00 | 50.00 |
| | Microcrystalline cellulose | 42.00 | 42.00 |
| | Croscarmellose sodium | 6.00 | 6.00 |
| Binding | Hydroxypropyl cellulose | 6.00 | 6.00 |
| solution | Purified water | 66.00 | 66.00 |
| Outside of | Vivapur112 | — | 34.00 |
| granule | Croscarmellose sodium | — | 8.00 |
| | Colloidal silicon dioxide | — | 2.00 |
| Lubricant | Magnesium stearate | 2.00 | 2.00 |
| | Total | 156.00 | 200.00 |

### Example 77

A mixture having the composition shown in Table 31 below was prepared with pharmaceutical additives.

TABLE 31

| | Example 77 |
|---|---|
| Components | mg/Dosage unit |
| Tegoprazan | 50.00 |
| Mannitol | 50.00 |
| Microcrystalline cellulose | 86.00 |
| Croscarmellose sodium | 10.00 |
| Colloidal silicon dioxide | 2.00 |
| Magnesium stearate | 2.00 |
| Total | 200.00 |

Specifically, tegoprazan was sieved together with mannitol, microcrystalline cellulose, croscarmellose sodium, and colloidal silicon dioxide in the amounts shown in Table 31 above, and then mixed. Sieved magnesium stearate was added to the mixture, and a lubrication process was performed to prepare a powdery mixture.

### Example 78

The immediate-release tablet shown in Table 32 below was prepared.

TABLE 32

| | | Example 78 |
|---|---|---|
| | Components | mg/Tablet |
| Uncoated tablet | Tegoprazan | 12.5 |
| | Mannitol | 67.5 |
| | Microcrystalline cellulose | 100.0 |
| | Croscarmellose sodium | 10.0 |
| | Hydroxypropyl cellulose | 6.0 |
| | Colloidal silicon dioxide | 2.0 |
| | Magnesium stearate | 2.0 |
| Coating | Opadry white | 6.0 |

| | |
|---|---|
| Total | 206.00 |

A granule and an uncoated tablet were prepared under substantially the same conditions and using the same method as in Examples 70 to 74, except that the components and contents shown in Table 32 above were used. Then, the prepared tablet was coated with Opadry white, thus preparing a final coated tablet.

### Example 79

The enteric-coated tablet shown in Table 33 below was prepared with pharmaceutical additives.

TABLE 33

| | | Example 79 |
|---|---|---|
| | Components | mg/Tablet |
| Uncoated tablet | Tegoprazan | 25.00 |
| | Mannitol | 25.00 |
| | Microcrystalline cellulose | 40.00 |
| | Croscarmellose sodium | 5.00 |
| | Hydroxypropyl cellulose | 3.00 |
| | Colloidal silicon dioxide | 1.00 |
| | Magnesium stearate | 1.00 |
| First coating | Hydroxypropyl methyl cellulose (3 cps) | 2.00 |
| | Polyethylene glycol 400 | 0.20 |
| Second coating | Methacrylic acid-ethyl acrylate copolymer | 7.67 |
| | Methacrylic acid copolymer S | 7.67 |
| | Triethyl citrate | 1.99 |
| | Polysorbate 80 | 0.05 |
| | Talc | 1.53 |
| Third coating | Hydroxypropyl methyl cellulose (3 cps) | 3.58 |
| | Polyethylene glycol 400 | 0.34 |

| | |
|---|---|
| Total | 125.03 |

An uncoated tablet was prepared under substantially the same conditions and using the same method as in Examples 70 to 74, except that the components and contents shown in Table 33 above were used. Then, using a tablet coater (Labcoat, O'hara), the prepared uncoated tablet was subjected sequentially to first coating, second coating and third coating using the components and contents shown in Table 33 above.

### Examples 80 to 83

In order to achieve various release patterns through combinations of the Examples, formulations, each including a tegoprazan immediate-release portion and a tegoprazan modified-release portion, were prepared using the combinations shown in Table 34 below.

TABLE 34

| | | | | | Example 82 | | Example 83 | |
|---|---|---|---|---|---|---|---|---|
| | Example 80 | | Example 81 | | (immediate- | | (immediate- | |
| | (immediate-release | | (immediate- | | release/enteric | | release/sustained- | |
| | tablet + enteric | | release/enteric | | sustained-release | | release bilayer | |
| | tablet) | | pellet capsule) | | pellet capsule) | | tablet) | |
| | | Weight | | weight | | weight | | weight |
| | Example | (mg) | Example | (mg) | Example | (mg) | Example | (mg) |
| Immediate-release | Example 78 | 206 | Example 76 | 50 | Example 76 | 50 | Example 76 | 50 |
| portion | | | | | | | | |
| (12.5 mg tegoprazan) | | | | | | | | |
| Modified-release | Example 79 | 125.03 | Example 21 | 93.5 | Example 62 | 89 | Example 72 | 100 |
| portion | | | | | | | | |
| (25 mg tegoprazan) | | | | | | | | |
| — | — | — | Hand | q.s. | Hand | q.s. | Opadry 85F | 4.5 |
| | | | capsule No. | | capsule No. | | | |
| | | | 3 | | 3 | | | |
| Total | — | — | — | 193.5 | — | 189 | — | 154.5 |

In order to confirm the immediate release and delayed release in the tablet form, Example 80 was prepared by combining an immediate-release tablet (Example 78) with an enteric tablet (Example 79). The immediate release/enteric pellet of Example 81 was a single formulation obtained by filling a hard capsule with the granule of Example 76 (an immediate release portion) and the enteric pellet of Example 21 (modified-release portion). The immediate-release/enteric sustained-release pellet of Example 82 was a single formulation obtained by filling a hard capsule with the granule of Example 76 (immediate-release portion) and the enteric sustained-release pellet of Example 62 (modified-release portion). The immediate-release/sustained-release tablet of Example 83 was a coated tablet prepared by tableting the granule of Example 76 and the granule of Example 72 into a bilayer tablet and then coating the bilayer tablet with Opadry 85F.

### Experimental Example 15. Dissolution Evaluation of Formulations

Dissolution of the formulations of Examples 80 to 83 was evaluated.

Dissolution evaluation was performed using a buffer transition dissolution test that can provide an environment similar to an in vivo environment.

Specifically, after the release pattern of the drug was examined using a 0.1N HCl solution (pH of about 1.1) for 2 hours, the acidity was increased to pH 7.4 by adding a buffer. Then, 0.5% polysorbate 80 was added, and the release rate of tegoprazan was comparatively evaluated.

Dissolution conditions were set to USP dissolution apparatus 2 (Paddle) and 50 rpm, and the sample solution obtained after the initiation of dissolution was analyzed using an ultraviolet spectrometer of high-performance liquid chromatography (HPLC; Agilent Technologies).

TABLE 35

| (min) | 5 | 10 | 15 | 30 | 45 | 60 | 90 | 120 | 135 | 150 | 165 | 180 | 210 | 240 | 360 | 480 | 600 | 840 |
|---|---|---|---|---|---|---|---|---|---|---|---|---|---|---|---|---|---|---|
| Example 80 | 30.4 | 32.3 | 32.9 | 32.8 | 32.4 | 31.7 | 30.5 | 31.5 | 31.9 | 41.5 | 63.0 | 74.9 | 79.6 | 81.5 | 84.4 | 85.1 | 85.8 | 86.5 |
| Example 81 | 27.8 | 30.4 | 31.4 | 32.1 | 32.5 | 32.6 | 31.2 | 31.7 | 39.9 | 55.1 | 64.5 | 71.1 | 76.4 | 78.7 | 80.6 | 80.9 | 81.0 | 80.9 |
| Example 82 | 26.2 | 28.6 | 29.1 | 29.6 | 29.7 | 29.7 | 28.8 | 30.2 | 33.0 | 36.9 | 39.9 | 42.2 | 46.2 | 50.1 | 57.6 | 63.5 | 66.7 | 70.0 |
| Example 83 | 24.3 | 30.1 | 34.0 | 41.4 | 47.4 | 48.7 | 57.1 | 63.8 | 73.7 | 74.6 | 75.7 | 76.8 | 77.5 | 78.3 | 80.8 | 81.6 | 82.8 | 85.1 |

Referring to the buffer transition dissolution test results in Table 35 above, it can be seen that, since all the Examples include an immediate-release portion and a modified-release portion, the active ingredient was rapidly released in the acidic solution and completely released in the weakly alkaline solution.

Specifically, when comparing Examples 80 to 83, it could be confirmed that the immediate-release/sustained-release bilayer tablet of Example 83, which is not an enteric concept, achieved sustained release together with immediate release under the acidic solution condition (within 2 hours). In addition, it could be confirmed, in case of Examples 80, 81 and 82 including the enteric concept, only the immediate-release portion was released under the acidic solution condition, and when the pH was changed to 6.8 by adding an additional buffer, the release of the remaining modified-release portion proceeded. In particular, it could be confirmed that, in the case of Example 82, the release did not increase rapidly even if the pH was changed, because the modified-release layer coating was added between the active ingredient layer and the enteric coating layer.

Therefore, it can be confirmed that the formulation of the present disclosure can release tegoprazan for a certain period in accordance with a desired release rate in a region ranging from gastric juice environment to the intestinal environment.

### Experimental Example 16. Evaluation of Pharmacokinetic Absorption Affect in Monkeys by Single Dose Parallel Design

For in vivo evaluation, non-clinical evaluation of the formulation combinations of Examples 80 to 83 was performed on 5 monkeys per group using a single dose parallel design method.

The non-clinical model animals used in the test were 6 cynomolgus monkeys (30 to 53 months old males with an average weight of 3.19±0.37 kg). Each test drug was administered orally to the animals by single-dose parallel design method in a fasting condition after fasting for at least 16 hours the day before administration (in the case of Example 80, the immediate-release tablet and the enteric tablet were administered simultaneously). At 0.5, 1, 1.5, 2, 3, 4, 5, 6, 8, 10, 12 and 24 hours, blood was collected from the femoral vein using a disposable syringe.

As a blood sample container, BD Microtainer® (Sodium Heparin tube, BD Biosciences, USA) was used. The collected blood was centrifuged at 3,000 rpm at 4° C. for 15 minutes to isolate plasma, and the plasma was stored in a cryogenic (−70° C.) freezer until analysis. The plasma concentration of the drug was analyzed LC-MS/MS in in-house method validation and electrospray ionization modes.

TABLE 36

| | | Example 81 | Example 82 | Example 83 | Example 80 |
|---|---|---|---|---|---|
| | K-CAB ®tablet | (IR/DR | (IR/DRSR | (IR/SR | (IR tablet/ |
| PK | 25 mg | capsule) | capsule) | bilayer) | DR tablet) |
| Tₘₐₓ(h) | 2.6 ± 1.1 | 4.2 ± 2.6 | 5.6 ± 0.5 | 3.0 ± 1.8 | 2.3 ± 1.5 |
| Cₘₐₓ(ng/mL) | 436 ± 138 | 523 ± 195 | 487 ± 92.7 | 592 ± 177 | 492 ± 103 |
| AUCₜ(ng · h/mL) | 2360 ± 588 | 3580 ± 314 | 3860 ± 750 | 3640 ± 1090 | 2820 ± 481 |

In the test results in Table 36 above, it was confirmed that the IR/DR, IR/DRSR and IR/SR formulations according to Examples 80 to 83 of the present disclosure had a Tₘₐₓ delay effect without affecting the total absorption, compared to the K-CAB® tablet or the IR capsule.

Therefore, it can be seen that the formulation of the present invention may achieve various release rate modifies not only the immediate-release portion but also by the delayed-release portion even in an actual in vivo environment. In addition, it can be seen that, since the formulation of the present disclosure can release tegoprazan sustainedly not only in the gastric juice environment but also in the intestinal environment, it is able to maintain a high blood concentration of the active ingredient for a long period of time after taking the formulation.

### Examples 84 to 87

As shown in Table 37 below, capsule formulations, each including a tegoprazan immediate-release portion and a tegoprazan modified-release portion, were prepared, which are combinations capable of double modified-release among the above-described embodiments.

TABLE 37

| | Example 84 | | Example 85 | | Example 86 | | Example 87 | |
|---|---|---|---|---|---|---|---|---|
| | | weight | | weight | | weight | | weight |
| | Example | (mg) | Example | (mg) | Example | (mg) | Example | (mg) |
| Immediate-release | Example 77 | 100.00 | Example 77 | 100.00 | Example 77 | 100.00 | Example 77 | 100.00 |
| portion | | | | | | | | |
| (25 mg tegoprazan) | | | | | | | | |
| Modified-release | Example 62 | 89.00 | Example 63 | 82.21 | Example 66 | 95.75 | Example 68 | 94.445 |
| portion | | | | | | | | |
| (25 mg tegoprazan) | | | | | | | | |

Examples 84 to 87 were single dosage forms prepared by double-filling a single capsule with the immediate-release portion (powder (mixture) of Example 77) and the modified-release portion (pellets of Examples 62, 63, 66 and 68, respectively).

### Experimental Example 17. Dissolution Evaluation of Combined Capsule Formulations

Dissolution tests for the capsule formulations were performed using a buffer transition dissolution test.

Specifically, after the release pattern of the drug was examined using a 0.1N HCl solution (pH of about 1.1) for 2 hours, the acidity was increased to pH 6.8 by adding a buffer. Then, 0.5% polysorbate 80 was added, and the release rate of tegoprazan was comparatively evaluated.

Dissolution conditions were set to USP dissolution apparatus 2 (Paddle) and 50 rpm, and the sample solution obtained after the initiation of dissolution was analyzed using an ultraviolet spectrometer of high-performance liquid chromatography (HPLC; Agilent Technologies). The results are shown in Table 38 below.

TABLE 38

| | 60 | 120 | 125 | 130 | 135 | 150 | 165 | 180 | 210 | 240 | 300 | 360 | 420 | 480 |
|---|---|---|---|---|---|---|---|---|---|---|---|---|---|---|
| | min | min | min | min | min | min | min | min | min | min | min | min | min | min |
| Example 84 | 48.6 | 48.2 | 48.9 | 52.8 | 58.5 | 68.2 | 72.7 | 75.7 | 79.1 | 81.3 | 85.5 | 88.2 | 93.8 | 96.1 |
| Example 85 | 48.3 | 47.8 | 47.0 | 47.7 | 47.8 | 52.9 | 69.6 | 79.1 | 88.6 | 92.8 | 96.2 | 96.9 | 101.4 | 101.1 |
| Example 86 | 47.3 | 47.2 | 47.3 | 47.3 | 47.2 | 47.1 | 47.4 | 48.2 | 49.1 | 52.1 | 82.5 | 100.3 | 100.8 | 100.6 |
| Example 87 | 47.9 | 48.2 | 48.4 | 48.1 | 47.9 | 47.8 | 48.3 | 53.8 | 86.0 | 95.4 | 98.5 | 98.8 | 98.5 | 98.4 |

Referring to the buffer transition dissolution test results in Table 38 above, it can be seen that the immediate-release portions of all the Examples were all dissolved within 1 hour in the acidic solution. When comparing the dissolution rate and the composition between the Examples, it could be confirmed that dissolution from the modified-release portion in the acidic solution did not occur in the case of Examples 84 to 87, and the dissolution occurred depending on the composition of each Example after pH adjustment (after 2 hours).

Therefore, since the formulation of the present disclosure includes an immediate-release portion and a modified-release portion, it may achieve not only rapid release of tegoprazan, but also delayed release and/or sustained release of tegoprazan, and thus may release tegoprazan in a sustained manner in a region ranging from the gastric juice environment to the intestinal environment.

As described above, the pharmaceutical composition of the present disclosure can prolong the therapeutic effect for a long time by modifying the release of the active ingredient, thus improving the patient's medication compliance. Thus, the composition may be effectively used for a disease for which a drug needs to be taken for a long period of time or the blood concentration of the drug at a time when the patient cannot take the drug needs to be maintained at a certain level or higher.

1. A modified-release pharmaceutical composition comprising: tegoprazan, an optical isomer thereof, a pharmaceutically acceptable salt thereof, a hydrate or solvate thereof, or a mixture thereof; and a release modifying agent.

2. The modified-release pharmaceutical composition of claim 1, wherein the release modifying agent comprises at least one selected from the group consisting of a sustained-release agent and an enteric agent.

3. The modified-release pharmaceutical composition of claim 1, wherein the pharmaceutical composition comprises a particle comprising, tegoprazan, an optical isomer thereof, a pharmaceutically acceptable salt thereof, a hydrate or solvate thereof, or a mixture thereof as an active ingredient.

4. The modified-release pharmaceutical composition of claim 3, wherein the release modifying agent is contained in the particle.

5. The modified-release pharmaceutical composition of claim 4, wherein the release modifying agent comprises at least one selected from the group consisting of a sustained-release agent and an enteric agent.

6. The modified-release pharmaceutical composition of claim 3, wherein the particle comprises a layer comprising the release modifying agent.

7. The modified-release pharmaceutical composition of claim 6, wherein the release modifying agent comprises at least one selected from the group consisting of a sustained-release agent and an enteric agent.

8. A modified-release pharmaceutical composition comprising: a core comprising tegoprazan, an optical isomer thereof, a pharmaceutically acceptable salt thereof, a hydrate or solvate thereof, or a mixture thereof as an active ingredient; and a release modifying agent-containing layer formed on the core.

9. The modified-release pharmaceutical composition of claim 8, wherein the core comprises: an inert particle; and an active ingredient layer, which comprises the active ingredient, positioned on the inert particle.

10. The modified-release pharmaceutical composition of claim 9, wherein the inert particle comprises at least one selected from the group consisting of white sugar, lactose, starch, mannitol, sucrose, dextrin, and microcrystalline cellulose.

11. The modified-release pharmaceutical composition of claim 8, wherein the pharmaceutical composition comprises an organic acid.

12. The modified-release pharmaceutical composition of claim 11, wherein the organic acid is at least one selected from the group consisting of tartaric acid, fumaric acid, succinic acid, citric acid, malic acid, glutamic acid and aspartic acid.

13. The modified-release pharmaceutical composition of claim 9, wherein the inert particle and the active ingredient are comprised in the core at a weight ratio within a range of 5:1 to 1:5.

14. The modified-release pharmaceutical composition of claim 8, wherein the core is a core tablet prepared by tableting a mixture of a granule comprising the active ingredient and a pharmaceutically acceptable additive.

15. The modified-release pharmaceutical composition of claim 8, wherein the core is a granule comprising a mixture comprising the active ingredient and a pharmaceutically acceptable additive.

16. The modified-release pharmaceutical composition of claim 8, wherein the release modifying agent comprises at least one selected from the group consisting of a sustained-release agent and an enteric agent.

17. The modified-release pharmaceutical composition of claim 16, wherein the enteric agent is any one or more selected from the group consisting of ethyl cellulose, cellulose acetate, polyvinyl acetate, cellulose butyrate phthalate, cellulose hydrogen phthalate, cellulose propionate phthalate, polyvinyl acetate phthalate, cellulose acetate phthalate, cellulose acetate trimellitate, hydroxypropyl methyl cellulose phthalate, polyvinyl acetate, hydroxypropyl methyl acetate, dioxypropyl methyl cellulose succinate, carboxymethyl ethyl cellulose, hydroxypropyl methyl cellulose acetate succinate, and polymers thereof; Shellac; and acrylic acid, methacrylic acid or esters thereof or copolymer formed from thereof.

18. The modified-release pharmaceutical composition of claim 17, wherein the enteric agent is any one or more selected from the group consisting of methacrylic acid-ethyl acrylate copolymer, methacrylic acid copolymer L, and methacrylic acid copolymer S.

19. The modified-release pharmaceutical composition of claim 18, wherein the enteric agent comprises methacrylic acid copolymer L and methacrylic acid copolymer S at a weight ratio within a range of 1:3 to 1:0.2.

20. The modified-release pharmaceutical composition of claim 18, wherein the enteric agent comprises methacrylic acid-ethyl acrylate copolymer and methacrylic acid copolymer S at a weight ratio within a range of 0.3:1 to 3:1.

21. The modified-release pharmaceutical composition of claim 16, wherein the sustained-release agent comprises one or more selected from the group consisting of polyvinyl alcohol, polyethylene oxide, methacrylic acid copolymer, hydroxypropyl methyl cellulose, ethyl cellulose, povidone, and talc.

22. The modified-release pharmaceutical composition of claim 8, wherein the release modifying agent-containing layer is pH-dependent soluble at pH 5.5 or higher.

23. The modified-release pharmaceutical composition of claim 8, wherein the release modifying agent-containing layer is comprised in an amount of 10 to 70 wt % based on the weight of the pharmaceutical composition.

24. (canceled)

25. The modified-release pharmaceutical composition of claim 8, wherein the core comprises the release modifying agent.

26. The modified-release pharmaceutical composition of claim 25, wherein the pharmaceutical composition comprises: a core comprising, tegoprazan, an optical isomer thereof, a pharmaceutically acceptable salt thereof, a hydrate or solvate thereof, or a mixture thereof as an active ingredient, and a first release modifying agent; and a release modifying agent-containing layer, which comprises a second release modifying agent, formed on the core, and wherein the first release modifying agent and the second release modifying agent each independently comprises at least one selected from the group consisting of a sustained-release agent and an enteric agent.

27. The modified-release pharmaceutical composition of claim 26, wherein the pharmaceutical composition further comprises an additional coating layer comprising a third release modifying agent, between the core and the release modifying agent-containing layer comprising the second release modifying agent.

28. The modified-release pharmaceutical composition of claim 27, wherein the first release modifying agent and the third release modifying agent comprise a sustained-release agent, and the second release modifying agent comprises an enteric agent.

29.-52. (canceled)
